(19) United States
(12) Patent Application Publication
(10) Pub. No.: US 2026/0219521 A1
(43) Pub. Date: Jul. 30, 2026

(54) COMPOUND, ELECTRO-OPTIC MATERIAL, ELECTRO-OPTIC MODULATOR, AND OPTOELECTRONIC INTEGRATED CIRCUITS

(71) Applicant: Samsung Electronics Co., Ltd., Suwon-si (KR)

(72) Inventors: Hyeong-Ju KIM, Suwon-si (KR); Kyung Bae PARK, Suwon-si (KR); Youngmok SON, Suwon-si (KR); Sungyoung YUN, Suwon-si (KR); Jiyoung JUNG, Suwon-si (KR); Sang Ho PARK, Suwon-si (KR); Hiromasa SHIBUYA, Suwon-si (KR)

(73) Assignee: Samsung Electronics Co., Ltd., Suwon-si (KR)

(21) Appl. No.: 19/367,399

(22) Filed: Oct. 23, 2025

(30) Foreign Application Priority Data
Jan. 24, 2025 (KR) 10-2025-0011952

(51) Int. Cl. G02F 1/00; C07D 213/57; C09K 11/06; G02F 1/21; G02F 1/225

(52) U.S. Cl. CPC G02F 1/0018; C07D 213/57; C09K 11/06; G02F 1/212; G02F 1/225; C09K 2211/1007; C09K 2211/1018

(57) ABSTRACT

A compound represented by Chemical Formula 1, an electro-optic material, an electro-optic modulator, and an optoelectronic integrated circuit are disclosed.

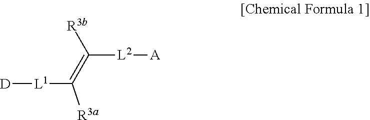

In Chemical Formula 1, A, D, L¹, L², R¹, R², R^{3a}, and R^{3b} are the same as described in the detailed description.

### CROSS-REFERENCE TO RELATED APPLICATION

This application claims priority to and the benefit of Korean Patent Application No. 10-2025-0011952 filed with the Korean Intellectual Property Office on Jan. 24, 2025, the entire contents of which are incorporated herein by reference.

### BACKGROUND

### 1. Field

The present disclosure relates to compounds, electro-optic materials, electro-optic modulators, and optoelectronic integrated circuits.

### 2. Description of the Related Art

An ultra-high-speed information and communications network may be based on an optical communication system that uses light as a medium for information transmission. Light may vibrate at ultra-high speeds through optical fibers and may travel at a much faster speed than electrons. In the optical communication system, electro-optic modulators may be used to convert digital data into optical signals, allowing data to be transmitted quickly and efficiently through optical fibers.

### SUMMARY

Silicon electro-optic modulators may convert digital data into optical signals by controlling silicon charge concentration. However, as the amount of data information transmitted increases, the controlling the silicon charge concentration may have a limitation in light modulation above a certain speed.

Some example embodiments provide a compound that is thermally stable and may be applied to an electro-optic modulator to improve optical modulation efficiency.

Some example embodiments provide an electro-optic material including the compound.

Some example embodiments provide an electro-optic modulator including the compound or the electro-optic material.

Some example embodiments provide an optoelectronic integrated circuit including the compound, the electro-optic material or the electro-optic modulator.

According to some example embodiments, a compound represented by Chemical Formula 1 may be provided.

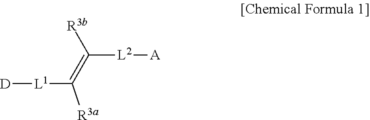

In Chemical Formula 1,
A may be a substituted or unsubstituted nitrogen cation-containing ring group,
D may be —NR¹R²,
L¹ and L² each independently may be a single bond, a substituted or unsubstituted C1 to C30 alkylene group, a substituted or unsubstituted C2 to C30 alkenylene group, a substituted or unsubstituted C6 to C30 arylene group, a substituted or unsubstituted divalent C2 to C30 heterocyclic group, a fused ring thereof, or any combination thereof,
R¹, R², R^{3a}, and R^{3b} each independently may be hydrogen, deuterium, a substituted or unsubstituted C1 to C20 alkyl group, a substituted or unsubstituted C1 to C20 alkylene group, a substituted or unsubstituted C2 to C30 alkenyl group, a substituted or unsubstituted C2 to C30 alkenylene group, a substituted or unsubstituted C1 to C30 alkoxy group, a substituted or unsubstituted C1 to C30 alkylthio group, a substituted or unsubstituted C6 to C30 aryl group, a substituted or unsubstituted C6 to C30 arylene group, a substituted or unsubstituted C3 to C30 heterocyclic group, a halogen, a hydroxyl group, a cyano group, or any combination thereof,
at least one of R^{3a} or R^{3b} may be a cyano group, and
R¹, R², R^{3a}, R^{3b}, and L¹ each independently may be present or two or three adjacent R¹, R², R^{3a}, R^{3b}, and L¹ may be linked to each other to form a ring.

In some example embodiments, A in Chemical Formula 1 may be a group represented by any one of Chemical Formulas 1a to 1c.

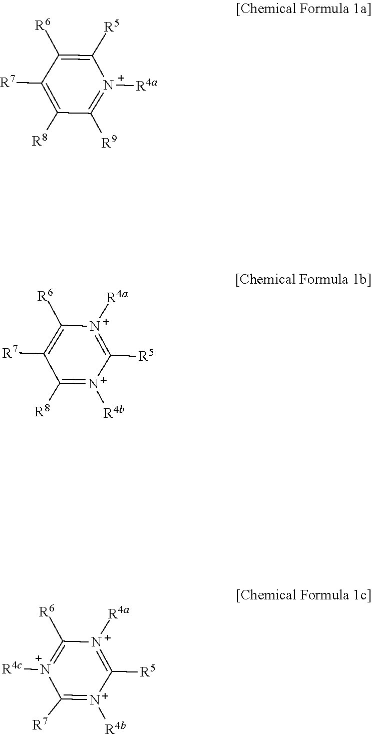

In Chemical Formulas 1a to 1c,
R^{4a}, R^{4b}, R^{4c}, and R⁵ to R⁹ may each independently be hydrogen, deuterium, a substituted or unsubstituted C1 to C20 alkyl group, a substituted or unsubstituted C2 to C30 alkenyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heterocyclic group, a halogen, a hydroxyl group, a cyano group, or any combination thereof, or a linking point with Chemical Formula 1,
R^{4a}, R^{4b}, R^{4c}, and R⁵ to R⁹ may each independently be present or two or three adjacent R^{4a}, R^{4b}, R^{4c}, and R⁵ to R⁹ may be linked to each other to form a ring, and
any one of R⁵ to R⁹ of Chemical Formula 1a, any one of R⁵ to R⁸ of Chemical Formula 1b, and any one of R⁵ to R⁷ of Chemical Formula 1c may each be a linking point with Chemical Formula 1.

In some example embodiments, L¹ and L² may each independently be a single bond, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene group, a substituted or unsubstituted dibenzotellurophenylene group, a fused ring thereof, or any combination thereof.

In some example embodiments, R¹ and R² may each independently be hydrogen, deuterium, a substituted or unsubstituted C1 to C20 alkyl group, a substituted or unsubstituted C1 to C20 alkylene group, a substituted or unsubstituted phenyl group, a substituted or unsubstituted phenylene group, a substituted or unsubstituted biphenyl group, a substituted or unsubstituted biphenylene group, a substituted or unsubstituted naphthyl group, a substituted or unsubstituted naphthylene group, or any combination thereof, wherein the “substituted” refers to replacement of at least one hydrogen by a hydroxy group, a silyl group, an amino group, a C1 to C30 alkyl group, a C6 to C30 aryl group, a C1 to C30 alkoxy group, a substituted or unsubstituted C1 to C30 alkylthio group, a C1 to C30 alkylseleno group, or any combination thereof, and R¹ and R² may each independently be present or two or three adjacent ones may be linked to each other to form a ring.

In some example embodiments, the compound may be represented by any one of Chemical Formulas 1-1 to 1-6.

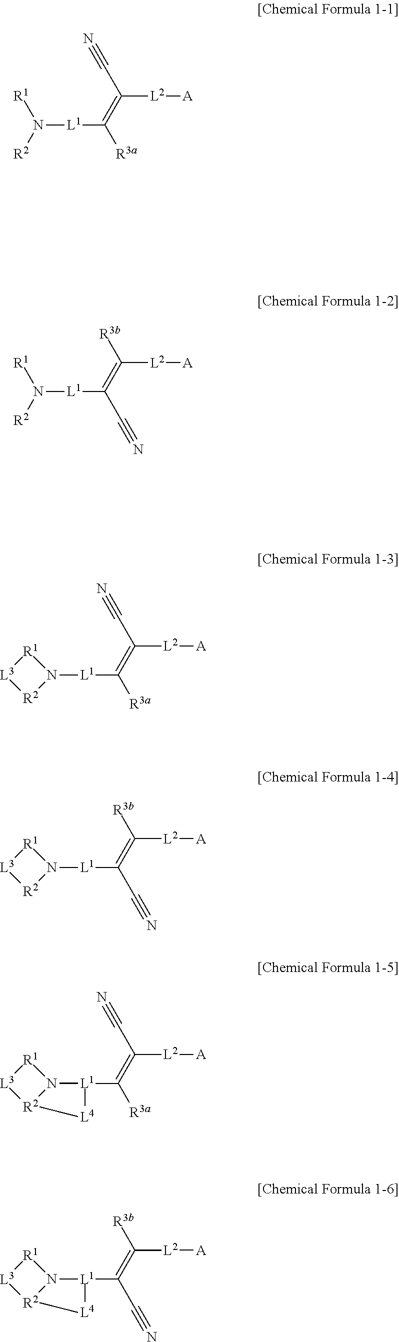

In Chemical Formulas 1-1 to 1-6,
A may be a substituted or unsubstituted nitrogen cation-containing ring group,
L¹ and L² may each independently be a single bond, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene group, a substituted or unsubstituted dibenzotellurophenylene group, a fused ring thereof, or any combination thereof,
R^{3a} or R^{3b} may be hydrogen, deuterium, or a substituted or unsubstituted C1 to C20 alkyl group,
R¹ and R² may each independently be hydrogen, deuterium, a substituted or unsubstituted C1 to C20 alkyl group, a substituted or unsubstituted C1 to C20 alkylene group, a substituted or unsubstituted phenyl group, a substituted or unsubstituted phenylene group, a substituted or unsubstituted biphenyl group, a substituted or unsubstituted biphenylene group, a substituted or unsubstituted naphthyl group, a substituted or unsubstituted naphthylene group, or any combination thereof,
L³ and L⁴ may each independently be a single bond, —O—, —S—, —Se—, —Te—, —NR^{a1}—, —BR^{a2}—, —SiR^{b}R^{c}—, —GeR^{d}R^{e}—, —(CR^{f}R^{g})ₙ₁—, —(C(R^{m})═C(Rⁿ))—, or —(C(R^{p})═N))—,
R^{a1}, R^{a2}, R^{b}, R^{c}, R^{d}, R^{e}, R^{f}, R^{g}, R^{m}, Rⁿ, and R^{p} may each independently be hydrogen, deuterium, a substituted or unsubstituted C1 to C20 alkyl group, a substituted or unsubstituted C1 to C20 alkoxy group, a substituted or unsubstituted C6 to C20 aryl group, or a halogen,
R^{a1}, R^{a2}, R^{b}, R^{c}, R^{d}, R^{e}, R^{f}, R^{g}, R^{m}, Rⁿ, and R^{p} may each independently be present or two adjacent ones may be linked to each other to form a ring, and
n1 may be 1 or 2.

In some embodiments, A in Chemical Formulas 1-1 to 1-6 may be a group represented by any one of the Chemical Formulas 1a to 1c.

According to some example embodiments, an electro-optic material may include the compound represented by Chemical Formula 1 and a polymer.

In some embodiments, an absorbance in a wavelength range of about 1300 nm to about 1700 nm of the polymer may be less than about 10%.

In some embodiments, the compound may further include a counterpart anion.

In some embodiments, the electro-optic material may include the compound and the polymer in a weight ratio of about 1:99 to about 99:1.

According to some example embodiments, an electro-optic modulator may include: a first electrode, a second electrode, and an electro-optic active layer between the first electrode and the second electrode, wherein the electro-optic active layer may include electro-optic molecules, and each of the electro-optic molecules may be the compound represented by the Chemical Formula 1.

In some embodiments, the compound may further include a counterpart anion, and the electro-optic active layer may further include a polymer.

In some embodiments, an absorbance in a wavelength range of 1300 nm to 1700 nm of the polymer may be less than 10%, and the electro-optic active layer may include the compound and the polymer in a weight ratio of 1:99 to about 99:1.

The electro-optic molecules in the electro-optic active layer may be aligned in one direction.

In some embodiments, the electro-optic modulator may further include a substrate including silicon, germanium, an III-V compound semiconductor substrate, an II-VI compound semiconductor, or any combination thereof.

According to some example embodiments, a method of manufacturing an electro-optic modulator may include: forming a first electrode, forming an electro-optic active layer on the first electrode and including electro-optic molecules, each of the electro-optic molecules being the compound represented by the Chemical Formula 1, forming a second electrode on the electro-optic active layer, and performing a poling process to align the electro-optic molecules.

In some embodiments, the performing of the poling process may include heat treating at a temperature of about +50° C. of the glass transition temperature of the polymer, applying a voltage to align the electro-optic molecules, and cooling.

In some embodiments, the electro-optic molecules may be aligned along a thickness direction of the electro-optic active layer.

According to some example embodiments, an optoelectronic integrated circuit may include: a substrate, an electrical signal processor on the substrate and configured to perform electrical signal processing to output an electrical signal, a light source on the substrate, an electro-optic modulator on the substrate and configured to output a modulated optical signal using the electrical signal output from the electrical signal processor and light supplied from the light source, and a photodetector on the substrate and configured to detect the modulated optical signal and convert the modulated optical signal into an electrical signal, wherein the electro-optic modulator includes a first electrode and a second electrode, and an electro-optic active layer between the first electrode and the second electrode, and the electro-optic active layer includes electro-optic molecules, each of the electro-optic molecules being the compound represented by the Chemical Formula 1.

In some embodiments, the substrate may include a silicon substrate, a germanium substrate, a silicon germanium substrate, an III-V compound semiconductor substrate, an II-VI compound semiconductor substrate, an SOI substrate, a SOG substrate, or any combination thereof.

In some embodiments, the light source may supply light of a wavelength within about 1000 nm to about 1700 nm.

According to some example embodiments, an optical communication device may include: an optoelectronic integrated circuit including an electrical signal processor, an electro-optic modulator, a photodetector, and a light source integrated therein, wherein the electro-optic modulator may include the compound represented by the Chemical Formula 1.

The compound may be thermally stable and may be applied to electro-optic modulators to improve optical modulation efficiency.

### BRIEF DESCRIPTION OF THE DRAWINGS

FIG. 1 is a schematic view showing an example of a Mach-Zehnder modulator according to some example embodiments,

FIG. 2 is an enlarged cross-sectional view of a portion of the electro-optic modulator in the Mach-Zehnder modulator of FIG. 1,

FIGS. 3A and 3B are cross-sectional views illustrating an example of a method of manufacturing an electro-optic modulator according to some example embodiments,

FIG. 4 is a schematic view showing an optoelectronic integrated circuit according to some example embodiments, and

FIG. 5 is a schematic view illustrating an example of a configuration diagram of an electronic device according to some example embodiments.

### DETAILED DESCRIPTION

Hereinafter, example embodiments of the present disclosure will be described in detail so that a person skilled in the art would understand the same. However, this disclosure may be embodied in many different forms and is not to be construed as limited to the example embodiments set forth herein.

In the drawings, the thickness of layers, films, panels, regions, etc., are exaggerated for clarity. Like reference numerals designate like elements throughout the specification. It will be understood that when an element such as a layer, film, region, or substrate is referred to as being “on” another element, it may be directly on the other element or intervening elements may also be present. In contrast, when an element is referred to as being “directly on” another element, there are no intervening elements present.

Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C” and “at least one of A, B, or C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., +10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., +10%) around the stated numerical values or shapes. When ranges are specified, the range includes all values therebetween such as increments of 0.1%.

As used herein, when a definition is not otherwise provided, “substituted” refers to replacement of a hydrogen atom of a compound by a substituent selected from a halogen atom, a hydroxy group, a silyl group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a silyl group, a C1 to C20 alkyl group, a C2 to C20 alkenyl group, a C2 to C20 alkynyl group, a C6 to C30 aryl group, a C7 to C30 arylalkyl group, a C1 to C30 alkoxy group, a C1 to C30 alkylthio group, a C1 to C30 alkylseleno group, a C1 to C20 heteroalkyl group, a C3 to C20 heteroaryl group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, and any combination thereof.

Hereinafter, as used herein, when a definition is not otherwise provided, “heterocyclic group” may be a C2 to C30 heterocyclic group. The heterocyclic group may refer to a ring group in which 1 to 3 carbons in at least one of a ring group selected from an aromatic hydrocarbon ring group (for example, a C6 to C30 arene group, a C6 to C20 arene group, or a C6 to C10 arene group, or a C6 to C30 aryl group, a C6 to C20 aryl group, or a C6 to C10 aryl group), an alicyclic hydrocarbon ring group (for example, a C3 to C30 cycloalkyl group, a C3 to C20 cycloalkyl group, or a C3 to C10 cycloalkyl group), and a fused ring group thereof are substituted with a heteroatom selected from N, O, S, P, and Si. Additionally, one or more carbon atoms of the heterocyclic group may be substituted with a thiocarbonyl group (C═S).

Hereinafter, as used herein, when a definition is not otherwise provided, “hetero” refers to including 1 to 4 heteroatoms selected from N, O, S, Se, Te, Si, and P.

Hereinafter, as used herein, when a definition is not otherwise provided, “aryl group” refers to a substituent in which all atoms of the cyclic functional group have p-orbitals and these p-orbitals form conjugation, and includes monocyclic, polycyclic, or fused ring polycyclic (e.g., rings that share adjacent pairs of carbon atoms) functional groups.

Hereinafter, as used herein, when a definition is not otherwise provided, “fused ring” may be a fused ring of two or more substituted or unsubstituted C5 to C30 hydrocarbon ring groups, a fused ring of two or more substituted or unsubstituted C2 to C30 heterocyclic groups, or a fused ring of a substituted or unsubstituted C5 to C30 hydrocarbon ring group and a substituted or unsubstituted C2 to C30 heterocyclic group (e.g., a fluorenyl group). Here, the hydrocarbon ring group and heterocyclic group are as defined above.

Hereinafter, the term “combination” refers to a mixture and a stacked structure of two or more.

As used herein, when specific definition is not otherwise provided, an energy level refers to the highest occupied molecular orbital (HOMO) energy level and the lowest unoccupied molecular orbital (LUMO) energy level.

Hereinafter, when specific definition is not otherwise provided, a work function or an energy level is expressed as an absolute value from a vacuum level. In addition, when the work function or energy level is referred to be deep, high, or large, it may have a large absolute value based on “0 eV” of the vacuum level while when the work function or energy level is referred to be shallow, low, or small, it may have a small absolute value based on “0 eV” of the vacuum level.

Hereinafter, as used herein, when a definition is not otherwise provided, the work functions and energy levels may be those calculated by Turbomole using the B3LYP/DGDZVP basis set.

As used herein, when a definition is not otherwise provided, “combination” of substituents may refer to substituents in which one substituent is substituted for another, or which exist fused to each other, or which are linked to each other by a single bond or a C1 to C10 alkylene group.

Hereinafter, a compound according to some example embodiments is described.

The compound may be an electro-optic molecule whose optical properties change in response to an electric field, and may be included in an electro-optic material as a component. For example, the compound may be a non-linear optical molecule that changes the intensity and phase of light in response to an electric field, or may be a chromophore molecule configured to absorb light of a desired and/or alternatively predetermined wavelength. Hereafter, the terms “compound”, “molecule”, and “electro-optic molecule” may be used interchangeably.

The compound may have a D-π-A structure including an electron donating unit D, pi-electron bridge π, and electron accepting unit A. The electron donating unit may be a relatively electron-rich moiety and may include a heteroatom such as nitrogen. The electron accepting unit may be a relatively electron-poor moiety (e.g., an electron-withdrawing moiety) and may be cationized. The pi-electron bridges may control intramolecular electron mobility between the electron donating unit and electron accepting unit.

As an example, the compound may be represented by Chemical Formula 1.

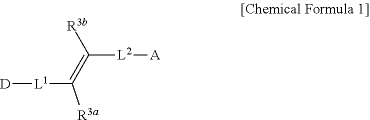

In Chemical Formula 1,
A may be a substituted or unsubstituted nitrogen cation-containing ring group, D may be —NR¹R²,
L¹ and L² may each independently be a single bond, a substituted or unsubstituted C1 to C30 alkylene group, a substituted or unsubstituted C2 to C30 alkenylene group, a substituted or unsubstituted C6 to C30 arylene group, a substituted or unsubstituted divalent C2 to C30 heterocyclic group, a fused ring thereof, or any combination thereof,
R¹, R², R^{3a}, and R^{3b} may each independently be hydrogen, deuterium, a substituted or unsubstituted C1 to C20 alkyl group, a substituted or unsubstituted C1 to C20 alkylene group, a substituted or unsubstituted C2 to C30 alkenyl group, a substituted or unsubstituted C2 to C30 alkenylene group, a substituted or unsubstituted C1 to C30 alkoxy group, a substituted or unsubstituted C1 to C30 alkylthio group, a substituted or unsubstituted C6 to C30 aryl group, a substituted or unsubstituted C6 to C30 arylene group, a substituted or unsubstituted C3 to C30 heterocyclic group, a halogen, a hydroxyl group, a cyano group, or any combination thereof,
at least one of R^{3a} or R^{3b} may be a cyano group, and
R¹, R², R^{3a}, R^{3b}, and L¹ may each independently be present or two or three adjacent R¹, R², R^{3a}, R^{3b}, and L¹ may be linked to each other to form a ring.

The compound represented by Chemical Formula 1 may have a D-π-A structure with a relatively high dipole moment u, and thus may form effective polarization in the molecule when an electric field is applied.

In addition, the compound represented by Chemical Formula 1 may increase intramolecular electron mobility between the electron donating unit D and the electron accepting unit A by including an ethylene group substituted with a cyano group (C≡N) in the pi-electron bridge π, and may also provide steric hindrance due to its bulky structure.

The steric hindrance may not only effectively limit and/or prevent aggregation between molecules, but also reduce electrostatic interactions between adjacent molecules, thereby increasing the orientational stability of molecules. For example, the compound may exhibit electro-optic activity by being polarized due to an electric field (e.g., poling) while exhibiting a non-centrosymmetric arrangement, and electrostatic interactions between molecules may disturb this ordered arrangement, thereby reducing the efficiency of converting the hyperpolarizability value (β_{zzz}) into electro-optic coefficients (r₃₃). The electro-optic coefficient (r₃₃) may refer to a degree to which the refractive index is changed by an electric field, and the larger the electro-optic coefficient (r₃₃), the stronger the electro-optic effect may be. The compound represented by Chemical Formula 1 may improve electro-optic properties by increasing the orientation stability of molecules by effectively reducing the electrostatic interactions between molecules due to the steric hindrance described above.

For example, A in Chemical Formula 1 may be a substituted or unsubstituted nitrogen cation-containing hexagonal ring group, and may be, for example, a group represented by any one of Chemical Formulas 1a to 1c.

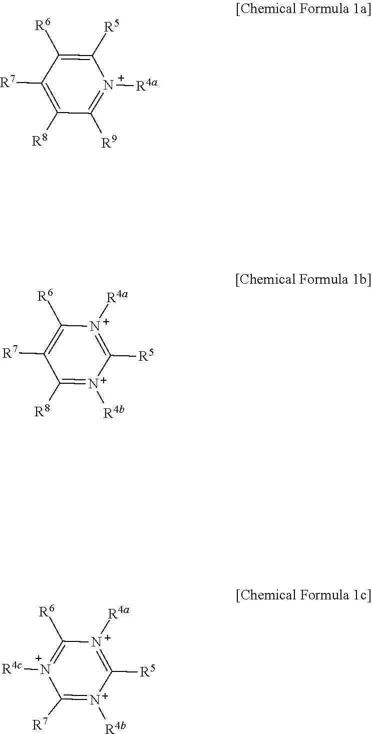

In Chemical Formulas 1a to 1c,
R^{4a}, R^{4b}, R^{4c}, and R⁵ to R⁹ may be each independently hydrogen, deuterium, a substituted or unsubstituted C1 to C20 alkyl group, a substituted or unsubstituted C2 to C30 alkenyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heterocyclic group, a halogen, a hydroxyl group, a cyano group, or any combination thereof, or a linking point with Chemical Formula 1,
R^{4a}, R^{4b}, R^{4c}, and R⁵ to R⁹ may each independently be present or two or three adjacent R^{4a}, R^{4b}, R^{4c}, and R⁵ to R⁹ may be linked to each other to form a ring, and
any one of R^{4a} and R⁵ to R⁹ of Chemical Formula 1a, any one of R^{4a}, R^{4b}, and R⁵ to R⁸ of Chemical Formula 1b, and any one of R^{4a}, R^{4b}, R^{4c}, and R⁵ to R⁷ of Chemical Formula 1c may each be a linking point with Chemical Formula 1.

For example, R⁷ of Chemical Formula 1a, 1b, or 1c may be a linking point with Chemical Formula 1.

For example, R⁶ or R⁸ of Chemical Formula 1a or 1b may be a linking point with Chemical Formula 1.

For example, R^{4a} of Chemical Formula 1a may be a linking point with Chemical Formula 1.

For example, R⁵ or R⁹ of Chemical Formula 1a may be a linking point with Chemical Formula 1.

For example, R^{4a} or R^{4b} of Chemical Formula 1b may be a linking point with Chemical Formula 1.

For example, R⁵ of Chemical Formula 1b may be a linking point with Chemical Formula 1.

For example, R^{4a}, R^{4b}, or R^{4c} of Chemical Formula 1c may be a linking point with Chemical Formula 1.

For example, R⁵, R⁶, or R⁷ of Chemical Formula 1c may be a linking point with Chemical Formula 1.

For example, two adjacent ones of R⁵ to R⁹ in Chemical Formula 1a may be linked to each other to form a benzene ring.

For example, two adjacent ones of R⁶ to R⁸ in Chemical Formula 1b may be linked to each other to form a benzene ring.

For example, R^{4a}, R^{4b}, and R^{4c} in Chemical Formula 1a, 1b, or 1c may each independently be hydrogen, deuterium, or a substituted or unsubstituted C1 to C20 alkyl group.

For example, L¹ and L² in Chemical Formula 1 may control a length of the pi-electron bridge (π), and may also control an overall length of the compound, the length between the electron donating group and electron accepting group, and the optical path length.

For example, L¹ and L² may be the same.

For example, L¹ and L² may be different from each other.

For example, L¹ and L² may each independently be a single bond, a substituted or unsubstituted ethylene group, a substituted or unsubstituted propylene group, a substituted or unsubstituted butylene group, a substituted or unsubstituted pentylene group, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene substituted group, a or unsubstituted dibenzotellurophenylene group, a fused ring thereof, or any combination thereof.

For example, at least one of L¹ or L² may be a single bond. For example, L¹ and L² may each be a single bond.

For example, at least one of L¹ or L² may include a substituted or unsubstituted polyene group (e.g., a substituted or unsubstituted ethylene group, a substituted or unsubstituted propylene group, a substituted or unsubstituted butylene group, or a substituted or unsubstituted pentylene group), thereby increasing the length of the double bond moiety in addition to the cyano group-substituted ethylene group in the pi-electron bridge (π), and thus increasing electron mobility in the compound.

For example, at least one of L¹ or L² may include a substituted or unsubstituted C6 to C30 arylene group, a substituted or unsubstituted divalent C2 to C30 heterocyclic group, a fused ring thereof, or any combination thereof.

For example, one of L¹ and L² may be a single bond, and the other of L¹ and L² may be a substituted or unsubstituted ethylene group, a substituted or unsubstituted propylene group, a substituted or unsubstituted C6 to C30 arylene group, a substituted or unsubstituted divalent C2 to C30 heterocyclic group, a fused ring thereof, or any combination thereof.

For example, L¹ and L² may each be a substituted or unsubstituted C6 to C30 arylene group. For example, one of L¹ and L² may be a substituted or unsubstituted phenylene group, and the other of L¹ and L² may be a substituted or unsubstituted phenylene group, a substituted or unsubstituted biphenylene group, or a substituted or unsubstituted terphenylene group.

For example, one of L¹ and L² may be a substituted or unsubstituted C6 to C30 arylene group, and one of L¹ and L² may be a substituted or unsubstituted divalent C2 to C30 heterocyclic group. For example, one of L¹ and L² may be a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, or a substituted or unsubstituted anthracenylene group, and the other of L¹ and

L² may be a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene group, or a substituted or unsubstituted dibenzotellurophenylene group.

For example, L¹ may include a substituted or unsubstituted divalent C2 to C30 heterocyclic group, and may include, for example, a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene group, or a substituted or unsubstituted dibenzotellurophenylene group.

For example, R^{3a} in Chemical Formula 1 may be a cyano group.

For example, R^{3b} in Chemical Formula 1 may be a cyano group.

For example, R^{3a} and R^{3b} in Chemical Formula 1 may each be a cyano group.

For example, one of R^{3a} and R^{3b} in Chemical Formula 1 may be a cyano group, and the other of R^{3a} and R^{3b} may be hydrogen, deuterium, or a substituted or unsubstituted C1 to C20 alkyl group.

For example, D in Chemical Formula 1 may be a substituted or unsubstituted amine group represented by —NR¹R², and for example, the substituents (R¹ and R²) may each independently be present, or may be a cyclic amine group in which the substituents (R¹ and R²) are linked to form a ring. Here, when R¹ and R² are each independently present, R¹ and R² may be monovalent groups, and when R¹ and R² are linked to each other or R¹ and/or R² are linked to adjacent R³ and/or L¹, R¹ and/or R² may be divalent groups.

For example, R¹ and R² may each independently be hydrogen, deuterium, a substituted or unsubstituted C1 to C20 alkyl group, a substituted or unsubstituted C1 to C20 alkylene group, a substituted or unsubstituted phenyl group, a substituted or unsubstituted phenylene group, a substituted or unsubstituted biphenyl group, a substituted or unsubstituted biphenylene group, a substituted or unsubstituted naphthyl group, a substituted or unsubstituted naphthylene group, or any combination thereof, and R¹ and R² may each independently be present, or two or three adjacent ones may be linked to each other to form a ring.

For example, at least one of R¹ or R² may be a substituted alkyl group, a substituted alkylene group, a substituted phenyl group, a substituted phenylene group, a substituted biphenyl group, a substituted biphenylene group, a substituted naphthyl group, or a substituted naphthylene group, wherein the “substituted” refers to replacement of at least one hydrogen by a hydroxy group, a silyl group, an amino group, a C1 to C30 alkyl group, a C6 to C30 aryl group, a C1 to C30 alkoxy group, a substituted or unsubstituted C1 to C30 alkylthio group, a C1 to C30 alkylseleno group, or any combination thereof.

For example, the compound represented by Chemical Formula 1 may be represented by any one of Chemical Formulas 1-1 to 1-6.

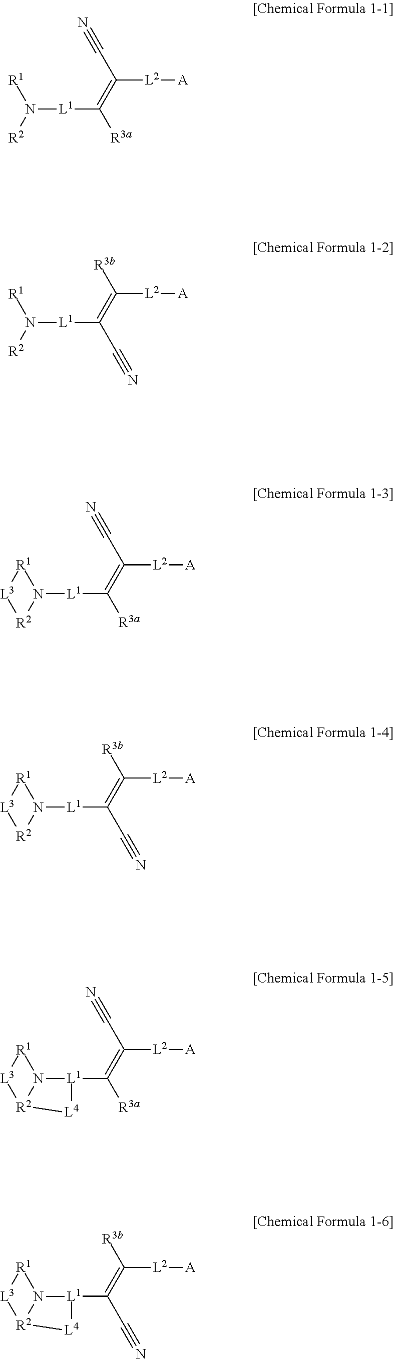

In Chemical Formulas 1-1 to 1-6,
A, L¹, L², R¹, R², R^{3a}, and R^{3b} may be the same as described above,
L³ and L⁴ may each independently be a single bond, —O—, —S—, —Se—, —Te—, —NR^{a1}—, —BR^{a2}—, —SiR^{b}R^{c}—, —GeR^{d}R^{e}—, —(CR^{f}R^{g})ₙ₁—, —(C(R^{m})═C(Rⁿ))—, or —(C(R^{p})═N))—,
wherein R^{a1}, R^{a2}, R^{b}, R^{c}, R^{d}, R^{e}, R^{f}, R^{g}, R^{m}, Rⁿ, and R^{p} may each independently be hydrogen, deuterium, a substituted or unsubstituted C1 to C20 alkyl group, a substituted or unsubstituted C1 to C20 alkoxy group, a substituted or unsubstituted C6 to C20 aryl group, or a halogen,
R^{a1}, R^{a2}, R^{b}, R^{c}, R^{d}, R^{e}, R^{f}, R^{g}, R^{m}, Rⁿ, and R^{p} are each independently present or two adjacent ones may be linked to each other to form a ring, and n1 may be 1 or 2.

For example, R¹ and R² of Chemical Formulas 1-1 to 1-6 may each independently be hydrogen, deuterium, a substituted or unsubstituted C1 to C20 alkyl group, a substituted or unsubstituted C1 to C20 alkylene group, a substituted or unsubstituted phenyl group, a substituted or unsubstituted phenylene group, a substituted or unsubstituted biphenyl group, a substituted or unsubstituted biphenylene group, a substituted or unsubstituted naphthyl group, a substituted or unsubstituted naphthylene group, or any combination thereof, and R¹ and R² are each independently present, or two or three adjacent ones may be linked to each other to form a ring. For example, at least one of R¹ or R² may be a substituted alkyl group, a substituted alkylene group, a substituted phenyl group, a substituted phenylene group, a substituted biphenylene group, a substituted biphenylene group, a substituted naphthyl group, or a substituted naphthylene group, wherein the “substituted” refers to replacement of at least one hydrogen by a hydroxy group, a silyl group, an amino group, a C1 to C30 alkyl group, a C6 to C30 aryl group, a C1 to C30 alkoxy group, a substituted or unsubstituted C1 to C30 alkylthio group, a C1 to C30 alkylseleno group, or any combination thereof.

The aforementioned compound may be a cation and may further include a counterpart anion.

The anion may be an inorganic or organic anion including boron (B), phosphorus (P), antimony (Sb), arsenic (As), a halogen ion (F⁻, Cl⁻, Br⁻, and/or I⁻), a nitric acid ion (NO₃⁻), and/or sulfuric acid ion (SO₄²⁻), but is not limited thereto. For example, the anion may be an inorganic borate, an organic borate, an inorganic phosphate, an organic phosphate, an inorganic antimonite, an organic antimonite, an inorganic arsenate, or an organic arsenate.

The inorganic borate, inorganic phosphate, inorganic antimonite, or inorganic arsenate may include, for example, boron (B), phosphorus (P), antimony (Sb) or arsenic (As), and a halogen ion (F⁻, Cl⁻, Br⁻, and/or I⁻), nitric acid ion (NO₃⁻) or sulfuric acid ion (SO₄²⁻), for example, BF₄⁻, PF₆⁻, SbF₆⁻, or AsF₆⁻, but is not limited thereto.

The organic borate, organic phosphate, organic antimonite, or organic arsenate may have a substituted or unsubstituted C1 to C20 alkyl group, a substituted or unsubstituted C6 to C20 aryl group or a substituted or unsubstituted C3 to C20 heteroaryl group linked to, for example, boron (B), phosphorus (P), antimony (Sb) or arsenic (As). The organic borate, organic phosphate, organic antimonite, or organic arsenate may be, for example, a borate, phosphate, antimonite, or arsenate including one or more halogen-substituted phenyl groups, but is not limited thereto.

The compound may be one of the compounds listed in Group 1, but is not limited thereto.

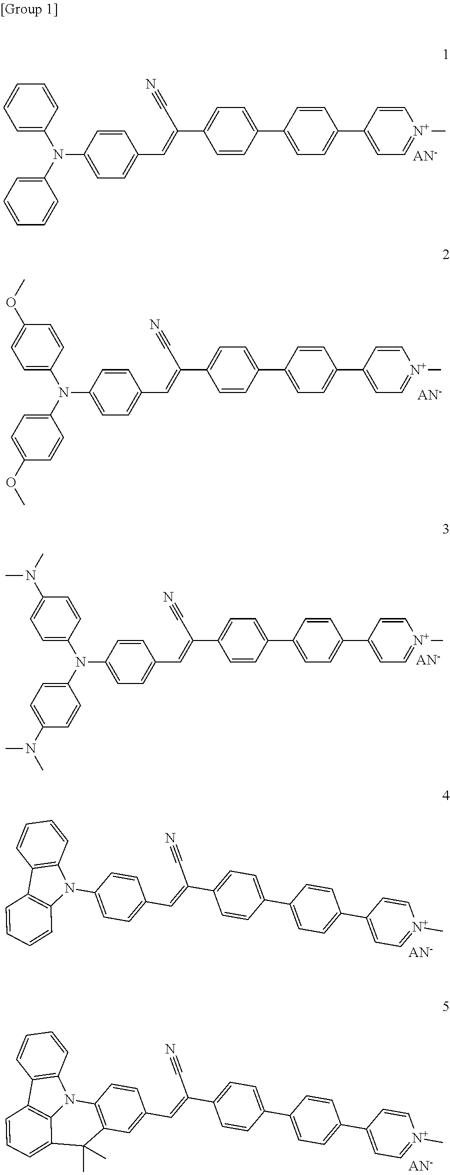

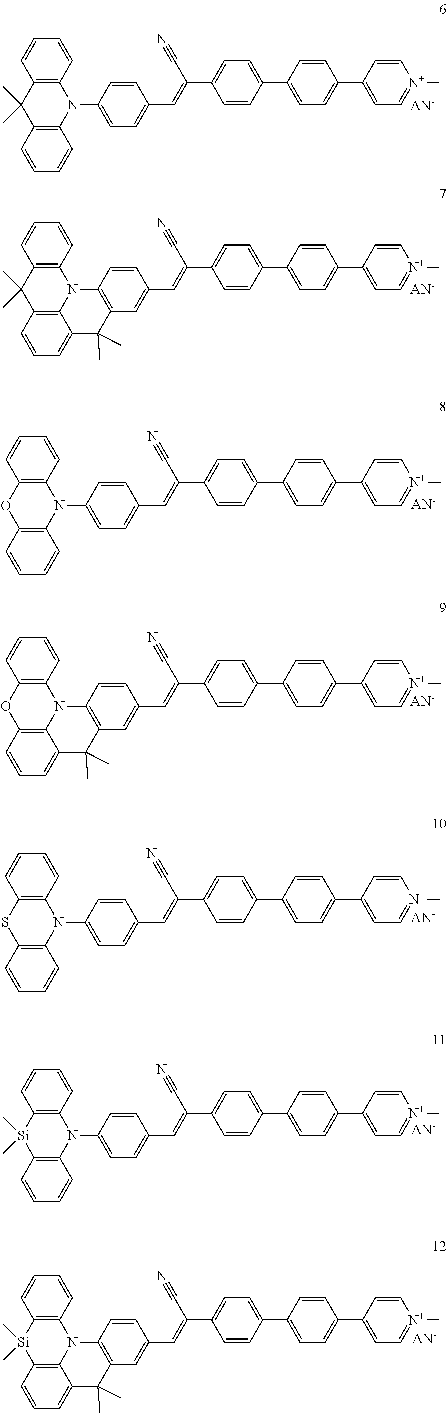

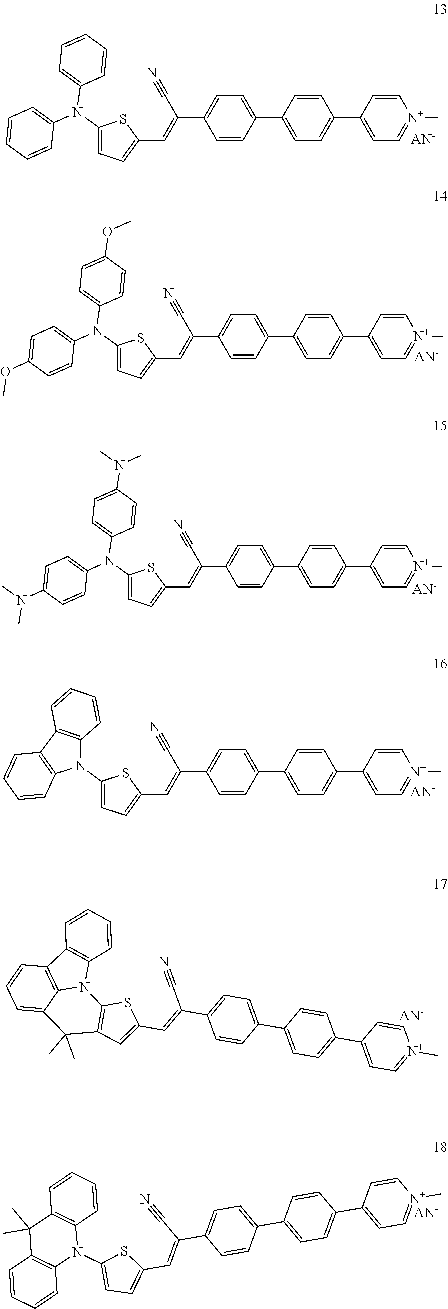

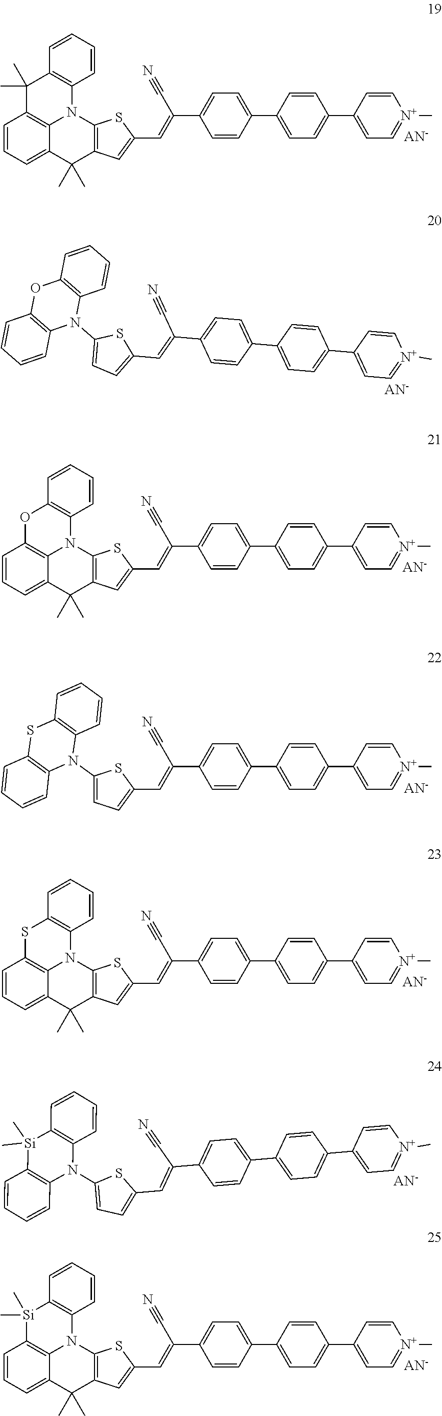

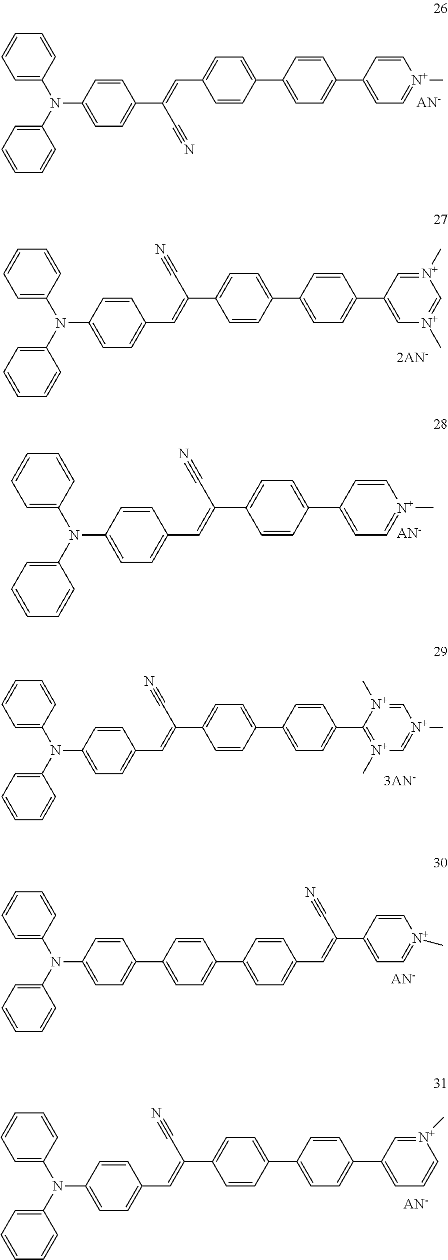 [SMILES: C[n+]1ccc(-c2ccc(-c3ccc(/C=C(\C#N)c4ccc(N(c5ccccc5)c5ccccc5)cc4)cc3)cc2)cc1 ; C[n+]1cc(-c2ccc(-c3ccc(/C(C#N)=C/c4ccc(N(c5ccccc5)c5ccccc5)cc4)cc3)cc2)c[n+](C)c1 ; C[n+]1ccc(-c2ccc(/C(C#N)=C/c3ccc(N(c4ccccc4)c4ccccc4)cc3)cc2)cc1 ; C[n+]1c[n+](C)c(-c2ccc(-c3ccc(/C(C#N)=C/c4ccc(N(c5ccccc5)c5ccccc5)cc4)cc3)cc2)[n+](C)c1 ; C[n+]1ccc(/C(C#N)=C/c2ccc(-c3ccc(-c4ccc(N(c5ccccc5)c5ccccc5)cc4)cc3)cc2)cc1 ; C[n+]1cccc(-c2ccc(-c3ccc(/C(C#N)=C/c4ccc(N(c5ccccc5)c5ccccc5)cc4)cc3)cc2)c1]

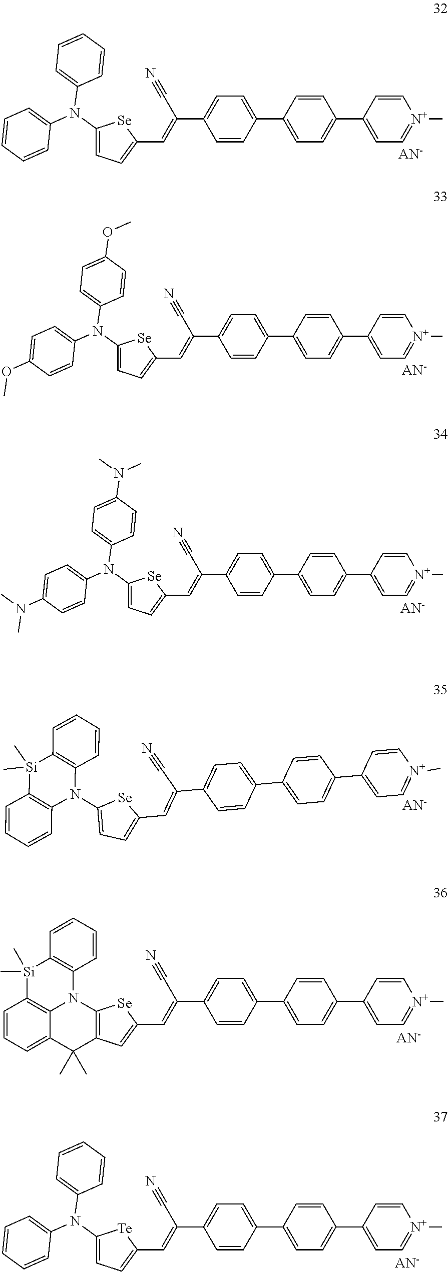

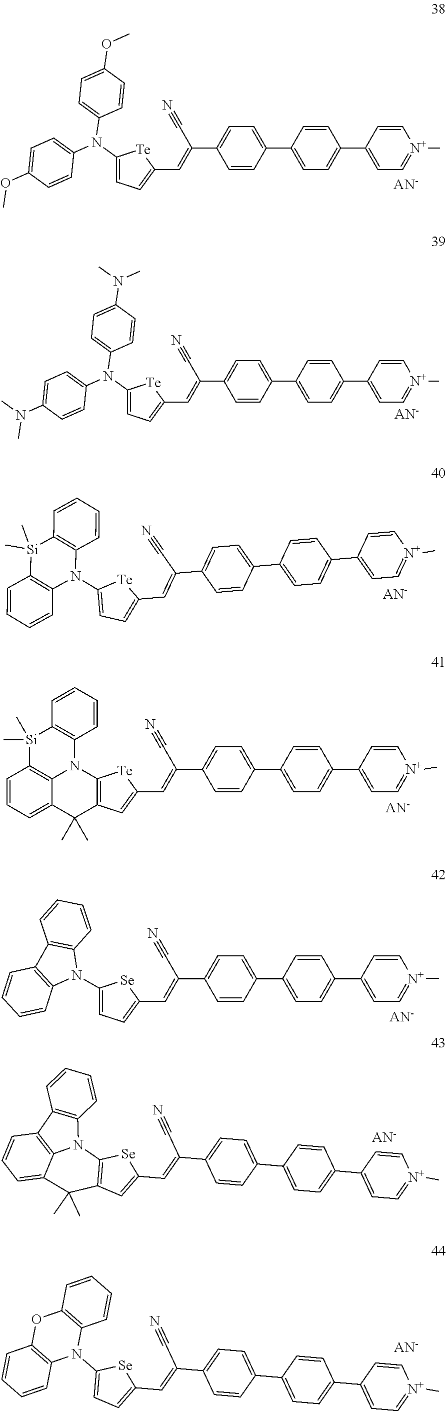

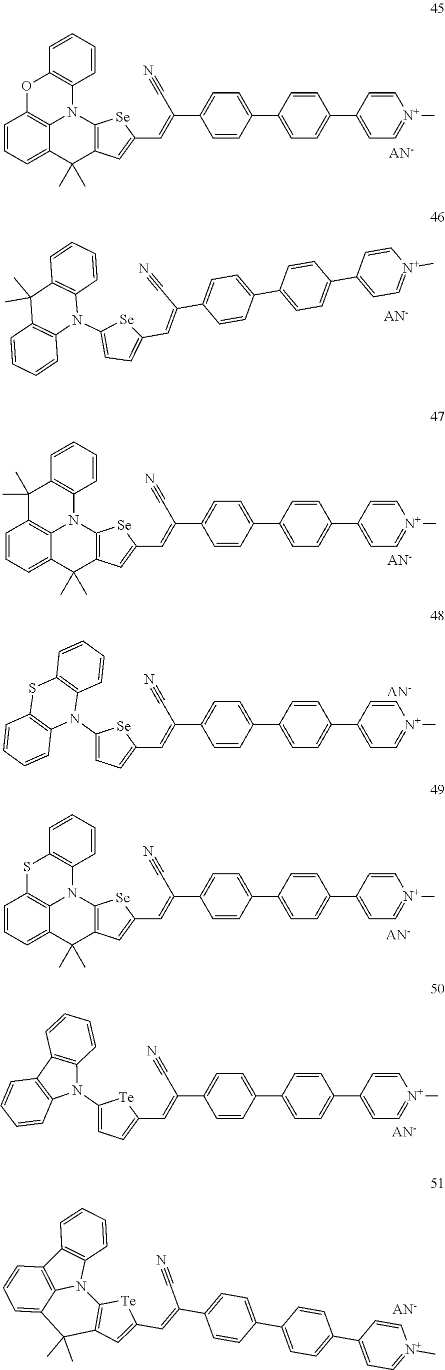

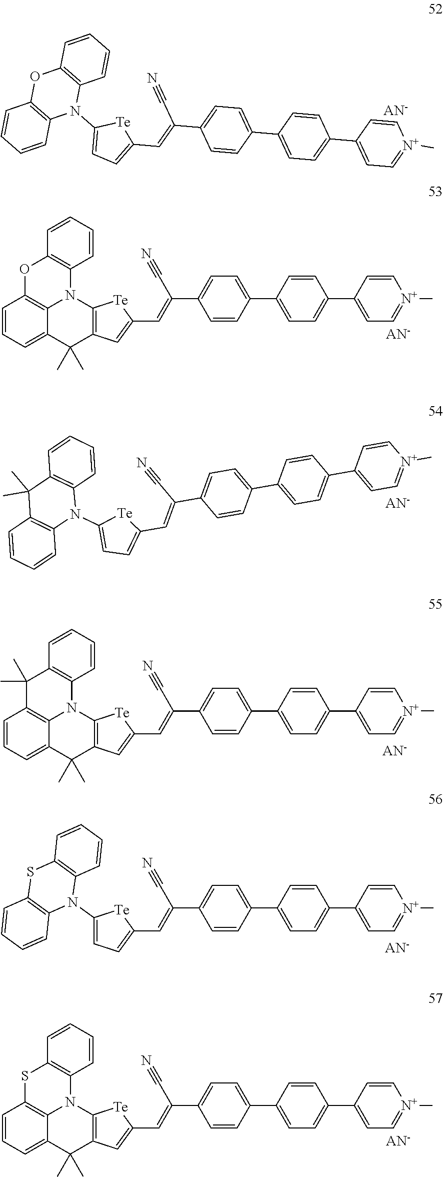

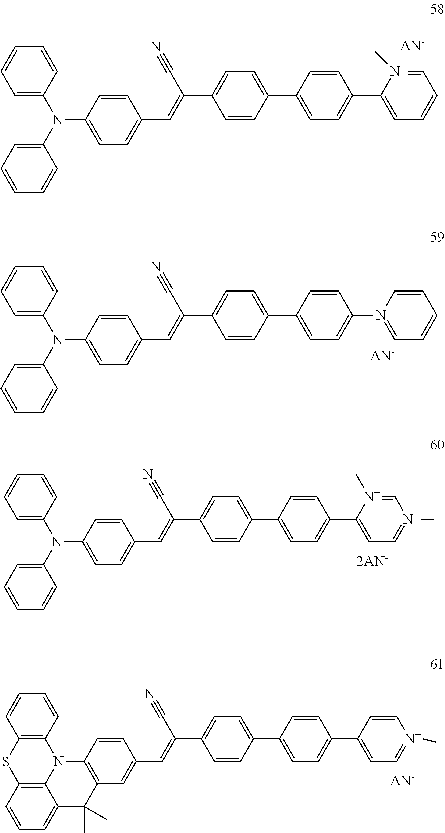 [SMILES: C[n+]1ccccc1-c1ccc(-c2ccc(/C(C#N)=C/c3ccc(N(c4ccccc4)c4ccccc4)cc3)cc2)cc1 ; N#C/C(=C\c1ccc(N(c2ccccc2)c2ccccc2)cc1)c1ccc(-c2ccc(-[n+]3ccccc3)cc2)cc1 ; C[n+]1ccc(-c2ccc(-c3ccc(/C(C#N)=C/c4ccc(N(c5ccccc5)c5ccccc5)cc4)cc3)cc2)[n+](C)c1 ; C[n+]1ccc(-c2ccc(-c3ccc(/C(C#N)=C/c4ccc5c(c4)C(C)(C)c4cccc6c4N5c4ccccc4S6)cc3)cc2)cc1]

In each compound of Group 1, AN⁻ may be a counterpart anion and is as described above.

The compound may be configured to absorb, for example, a portion of light in the visible light wavelength range, for example, at least a portion of light in the wavelength range of about 400 nm to about 750 nm.

The compound may not be configured to absorb light in the wavelength range of, for example, about 1300 nm to about 1700 nm.

The compound may have a relatively high glass transition temperature T_{g}, for example, may have a glass transition temperature of greater than or equal to about 100° C. By having a glass transition temperature within the above range, the compound may exhibit high thermal stability and orientational stability, and for example, the compound may reduce or prevent an ordered arrangement that has been aligned by an electric field from changing into a disordered isotropic arrangement due to electrostatic interactions in a relatively wide temperature range (for example, a temperature range lower than the glass transition temperature of the compound). Within the above range, the glass transition temperature of the compound may be about 100 to about 500° C.

The compound may have a relatively high dipole moment (μ). For example, the dipole moment of the compound may be greater than or equal to about 10 Debye, within the above range, greater than or equal to about 20 Debye, greater than or equal to about 25 Debye, greater than or equal to about 30 Debye, greater than or equal to about 35 Debye, or greater than or equal to about 40 Debye, within the above range, about 15 to about 100 Debye, about 20 to about 100 Debye, about 25 to about 100 Debye, about 30 to about 100 Debye, about 35 to about 100 Debye, or about 40 to about 100 Debye.

The compound may have a relatively high polarization rate (α). For example, the polarization rate (α) of the compound may be greater than or equal to about 500, and within the above range, about 500 to about 5,000.

The compound may have a relatively high hyperpolarizability (β). For example, the hyperpolarizability (β) of the compound may be greater than or equal to about 100, within the above range, greater than or equal to about 200, greater than or equal to about 300, greater than or equal to about 500, greater than or equal to about 700, greater than or equal to about 1,000, or greater than or equal to about 1,200, within the above range, about 100 to about 10,000, about 200 to about 10,000, about 300 to about 10,000, about 500 to about 10,000, about 700 to about 10,000, about 1,000 to about 10,000, or about 1,200 to about 10,000.

Here, the dipole moment (μ), polarization rate (α), and hyperpolarizability (β) may be calculated using density functional theory (DFT) and may be the values calculated using, for example, B3LYP/DGDZVP, M06-2X/Def2TZVP.

The compound may have relatively high electro-optic coefficients (r₃₃) by having relatively high hyperpolarizability (β) as described above. The electro-optic coefficient (r₃₃) may be Pockel's coefficient and may be substantially proportional to the hyperpolarizability (β). For example, the electro-optic coefficient (r₃₃) of the compound may be greater than or equal to about 5 pm/V, within the above range, about 5 to about 2000 pm/V, about 5 to about 1500 pm/V, about 5 to about 1000 pm/V, about 5 to about 800 pm/V, about 5 to about 600 pm/V, about 5 to about 500 pm/V, about 10 to about 2000 pm/V, about 10 to about 1500 pm/V, about 10 to about 1000 pm/V, about 10 to about 800 pm/V, about 10 to about 600 pm/V, or about 10 to about 500 pm/V.

The compound may be non-linear optical molecule and/or chromophore molecule and may be used alone or in combination with other materials as electro-optic materials in devices or components required electro-optic control.

As an example, the compound may be used alone as an electro-optic material. The electro-optic material may be one type of the compound or a mixture of two or more types of the compounds.

As an example, the compound may be used as an electro-optic material together with a polymer.

For example, the electro-optic material may be a mixture of the compound (electro-optic molecules) and a polymer, and for example, the electro-optic molecules may be doped into a polymer (polymer matrix). The electro-optic molecules may be included in one or more types. During an electric field application (e.g., poling) and a heat treatment process (e.g., heat treatment at a temperature of about ±50° C. of the glass transition temperature of the polymer), the electro-optic molecules may be aligned in one direction and fixed in the polymer matrix.

For example, the electro-optic material may be in a form in which a structural unit derived from the electro-optic molecules is linked to a polymer, and may include, for example, a structural unit derived from the electro-optic molecules in the main chain or side chain of the polymer. The electro-optic molecules may be in one or more types.

For example, the electro-optic material may be in a form in which adjacent polymer backbones are crosslinked through a structural unit derived from the electro-optic molecules. The compound may be in one or more types, and may further include a thermal crosslinking agent and/or a photo-crosslinking agent. For example, during the application of an electric field (e.g., poling) and heat treatment, the electro-optic molecules may be crosslinked between adjacent polymer backbones and aligned and fixed in one direction.

The polymer may be selected from insulating polymers that do not affect the electro-optic properties of the electro-optic molecules, and for example, may be selected from insulating polymers that do not substantially absorb light of the light absorption wavelength region of the electro-optic molecules. For example, the absorbance at a wavelength (e.g., the wavelength of the light source) at which the electro-optic effect of the insulating polymer is required may be less than about 10%, and within this range, may be 0 to about 8%, 0 to about 6%, 0 to about 5%, 0 to about 3%, 0 to about 2% or 0 to 1%.

The wavelength (e.g., the wavelength of the light source) at which the electro-optical effect is required may be in the range of about 1000 nm to about 1700 nm (e.g., about 1300 nm to about 1700 nm), but is not limited thereto.

The polymer may have a relatively high glass transition temperature T_{g}, and for example, the glass transition temperature T_{g} of the polymer may be greater than or equal to about 30° C., and within this range, may be about 30 to about 300° C., about 50 to about 300° C., or about 80 to about 300° C. For example, the polymer may be poly(methyl methacrylate) (PMMA), polycarbonate (PC) (e.g., amorphous polycarbonate), a copolymer thereof, a derivative thereof, or any combination thereof, but is not limited thereto.

The electro-optic molecules and the polymer may be included in a weight ratio of about 1:99 to about 99:1, and within that range, about 5:95 to about 95:5, about 10:90 to about 90:10, about 20:80 to about 80:20, about 30:70 to about 70:30, about 40:60 to about 60:40, or about 50:50. For example, the electro-optic molecules may be included in an equal or lesser amount than the polymer, and for example, the electro-optic molecules and the polymer may be included in a ratio of about 50:50 to about 1:99.

The compound (electro-optic molecules) or electro-optic material may be included to a device or component required electro-optic control. As an example, the compound (electro-optic molecules) or electro-optic material may be included in an electro-optic modulator.

An electro-optic modulator according to some example embodiments is described below. Here, a Mach-Zehnder modulator is described as an example.

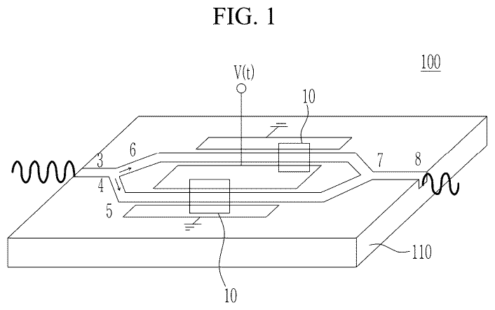

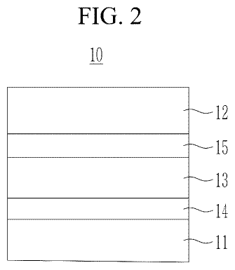

FIG. 1 is a schematic view showing an example of a Mach-Zehnder modulator according to some example embodiments, and FIG. 2 is an enlarged cross-sectional view of a portion of an electro-optic modulator in the Mach-Zehnder modulator of FIG. 1.

Referring to FIG. 1, a Mach-Zehnder modulator 100 according to some example embodiments includes an input section 3, a branch section 4, a first optical waveguide 5, a second optical waveguide 6, a combining section 7, and an output section 8.

The Mach-Zehnder modulator 100 may be supported by a substrate 110. The substrate 110 may be an inorganic substrate such as glass or an inorganic semiconductor (e.g., silicon, germanium, or a compound semiconductor); a polymer substrate including polycarbonate, polymethylmethacrylate, polyethylene terephthalate, polyethylene naphthalate, polyimide, polyamide, polyamideimide, polyethersulfone, or any combination thereof; or a semiconductor substrate such as a wafer or a semiconductor compound. For example, the substrate may include a semiconductor such as silicon, germanium, an III-V compound semiconductor, an II-VI compound semiconductor, or any combination thereof, but is not limited thereto.

The input section 3 may be a light source or may be connected to a light source, and the light supplied from the light source may be branched into a first optical waveguide 5 and a second optical waveguide 6 at the branch section 4. The first optical waveguide 5 and/or the second optical waveguide 6 include an electro-optic modulator 10.

Referring to FIG. 2, the electro-optic modulator 10 includes a first electrode 11, a second electrode 12, an electro-optic active layer 13, and auxiliary layers 14 and 15.

One of the first electrode 11 or the second electrode 12 may be an input electrode and the other may be a reference electrode. The first electrode 11 and the second electrode 12 may each independently include, for example, a metal, an oxide conductor, a carbon conductor, or any combination thereof. The metal may be, for example, aluminum (AI), magnesium (Mg), silver (Ag), gold (Au), nickel (Ni), molybdenum (Mo), tungsten (W), magnesium-silver (Mg—Ag), magnesium-aluminum (Mg—Al), alloys thereof, or any combination thereof. The oxide conductor may be, for example, one or more selected from indium tin oxide (ITO), indium zinc oxide (IZO), zinc tin oxide (ZTO), aluminum tin oxide (ATO), and aluminum zinc oxide (AZO), and the carbon conductor may be one or more selected from graphene and carbon nanostructure.

The electro-optic active layer 13 may include an electro-optic material whose optical properties change according to an electric field, and the electro-optic material is as described above. For example, the electro-optic material may be a mixture of the compound (electro-optic molecules) and a polymer. The polymer may be polymethyl methacrylate (PMMA), polycarbonate (PC), a copolymer thereof, or any combination thereof, but is not limited thereto. A weight ratio of the electro-optic molecules to the polymer may be about 1:99 to about 99:1, and within the above range, may be about 5:95 to about 95:5, about 10:90 to about 90:10, about 20:80 to about 80:20, about 30:70 to about 70:30, about 40:60 to about 60:40 or about 50:50.

The electro-optic molecules may be aligned in one direction in the electro-optic active layer 13, for example, may be aligned along the thickness direction of the electro-optic active layer 13. The alignment of the electro-optic molecules may be carried out by heat treatment (e.g., heat treatment at a temperature of about +50° C. of the glass transition temperature of the polymer) and a poling process, which will be described later.

The auxiliary layers 14 and 15 may be between the first electrode 11 and the electro-optic active layer 13 and between the second electrode 12 and the electro-optic active layer 13, and may be, for example, a charge transport layer and/or a charge blocking layer. The auxiliary layers 14 and 15 may include a metal oxide, a semi-metal oxide, an organic material, or any combination thereof, but is not limited thereto. At least one of the auxiliary layers 14 and 15 may be omitted.

When light supplied from a light source passes through the first optical waveguide 5 and/or the second optical waveguide 6, the electro-optic modulator 10 may modulate optical properties by an electric field applied to an input electrode, which is one of the first electrode 11 or the second electrode 12, and emit modulated light.

The modulated light passing through the first optical waveguide 5 and the second optical waveguide 6 may be combined in the combining section 7 and may be output a modulated light output signal with a desired and/or alternatively predetermined intensity modulation into the output unit 8.

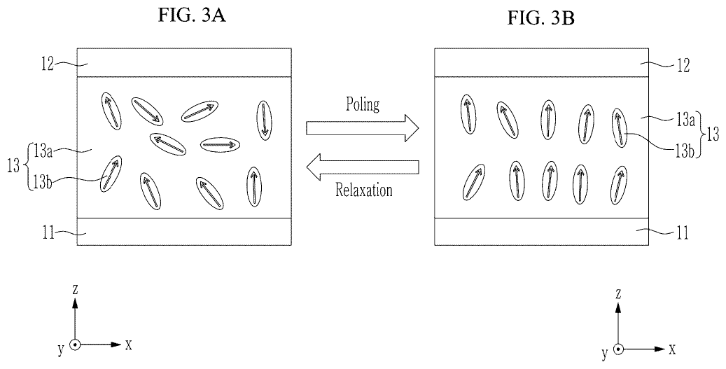

Hereinafter, an example of a method of manufacturing the electro-optic modulator 10 according to some example embodiments is described with reference to FIGS. 3A and 3B together with FIG. 2.

FIGS. 3A and 3B are cross-sectional views illustrating a method of manufacturing an electro-optic modulator according to some example embodiments.

First, the first electrode 11 is formed, and the first electrode 11 may be formed by depositing, for example, a metal, an oxide conductor, a carbon conductor, or any combination thereof.

Subsequently, a solution for an electro-optic active layer 13 is coated on the first electrode 11 (or auxiliary layer 14) to form an electro-optic active layer 13. The coating may be spin coating, slit coating, dip coating, inkjet coating, or any combination thereof, but is not limited thereto.

The solution for the electro-optic active layer 13 may include a polymer 13a and electro-optic molecules 13b, each of the electro-optic molecules may be the compound represented by Chemical Formula 1. In some embodiments, each of the electro-optic molecules may be the compound represented by any one of the Chemical Formulas 1-1 to 1-6.

The polymer 13a may be, for example, polymethyl methacrylate (PMMA), polycarbonate (PC) (e.g., amorphous polycarbonate), a copolymer thereof, a derivative thereof, or any combination thereof, but is not limited thereto. The electro-optic molecules 13b may include a cation represented by Chemical Formula 1 (or any one of the Chemical Formulas 1-1 to 1-6) and counterpart anion. The electro-optic molecules 13b may be dispersed in the polymer 13a. The solution for the electro-optic active layer 13 may include the electro-optic molecules 13b and the polymer 13a in a weight ratio of about 1:99 to about 99:1, and within the above range, may include the electro-optic molecules 13b and the polymer 13a in a weight ratio of about 5:95 to about 95:5, about 10:90 to about 90:10, about 20:80 to about 80:20, about 30:70 to about 70:30, about 40:60 to about 60:40, or about 50:50. For example, the electro-optic molecules 13b may be included in an amount equal to or less than that of the polymer 13a, and for example, the electro-optic molecules 13b and the polymer 13a may be included in a ratio of about 50:50 to about 1:99.

Subsequently, the second electrode 12 (or an auxiliary layer 15 and a second electrode 12) may be formed on the electro-optic active layer 13. The second electrode 12 may be formed by depositing, for example, a metal, an oxide conductor, a carbon conductor, or any combination thereof.

Then, a poling process is performed to align the electro-optic molecules 13b in the electro-optic active layer 13. The poling process may include heat treating the electro-optic active layer 13, applying a voltage to align the electro-optic molecules 13b, and cooling.

The heat treatment may be performed on the electro-optic active layer 13 at a temperature of about +50° C. of the glass transition temperature T_{g} of the polymer 13a. The heat treatment may be performed within a temperature range from −40° C. of the glass transition temperature T_{g} of the polymer 13a to about 40° C. of the glass transition temperature T_{g} of the polymer 13a, but is not limited thereto. The heat treatment may be performed within a temperature range from −30° C. of the glass transition temperature T_{g} of the polymer 13a to about 30° C. of the glass transition temperature T_{g} of the polymer 13a, but is not limited thereto. By such a heat treatment, the solution for the electro-optic active layer 13 may be softened, and accordingly, the electro-optic molecules 13b in the polymer 13a may move or rotate freely, as shown in FIG. 3A.

Then, by applying an electric field to the first electrode 11 and the second electrode 12, as shown in FIG. 3B, the electro-optic molecules 13b may be aligned along one direction, and for example, may be aligned substantially parallel to the thickness direction (e.g., z direction) of the electro-optic active layer 13. For example, the aligned direction of the electro-optic molecules 13b may be within about ±20 degrees, about ±10 degrees, or about ±5 degrees with respect to the thickness direction (e.g., z direction) of the electro-optic active layer 13, but is not limited thereto.

Then, by cooling to room temperature and removing the electric field, the electro-optic active layer 13 including the electro-optic molecules 13b aligned in one direction may be formed. The electro-optic molecules 13b in the electro-optic active layer 13 may be chromophore molecules configured to absorb light and may be a non-linear optical material that changes the intensity and phase of light by polarization.

The electro-optic modulator may be manufactured as an optoelectrical integrated circuit together with other components. The optoelectronic integrated circuit may have various components, including the electro-optic modulators, integrated together on a substrate.

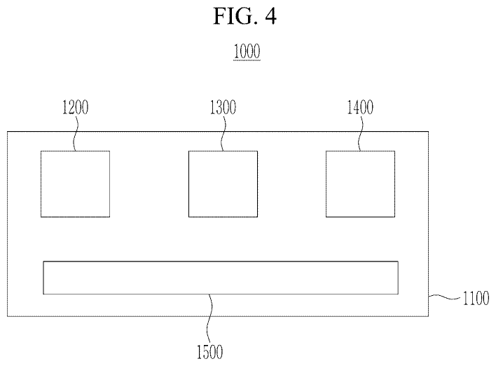

FIG. 4 is a schematic view showing an optoelectronic integrated circuit according to some example embodiments.

Referring to FIG. 4, the optoelectronic integrated circuit 1000 includes

a substrate 1100, a light source 1200, an electro-optic modulator 1300, a photodetector 1400, and an electrical signal processor 1500.

The substrate 1100 may include a silicon substrate, a germanium substrate, a silicon germanium substrate, an III-V compound semiconductor substrate, an II-VI compound semiconductor substrate, an SOI substrate, a SOG substrate, or any combination thereof, and may be, for example, a CMOS substrate.

The light source 1200 is disposed on the substrate 1100 and may include, for example, a laser diode. The laser diode may supply light of a desired and/or alternatively predetermined wavelength to an electro-optic modulator 1300. For example, the laser diode may supply light of a wavelength ranging from about 1000 nm to about 1700 nm, or may supply light of a wavelength ranging from about 1300 nm to about 1700 nm, or within that range, may supply light of a wavelength of, for example, about 1550 nm or about 1300 nm. For example, a laser diode may supply light of a wavelength of greater than or equal to about 400 nm and less than about 1000 nm, and may supply light of a wavelength of about 850 nm.

The electro-optic modulator 1300 is disposed on the substrate 1100 and may include the Mach-Zehnder modulator 100 (including the electro-optic modulator 10). The electro-optic modulator 1300 may output a modulated optical signal using an electrical signal output from the electrical signal processor 1500 and light supplied from the light source 1200.

The photodetector 1400 is disposed on the substrate 1100 and may be configured to detect the modulated optical signal from an electro-optic modulator 1300, convert the modulated optical signal into an electrical signal, and output the electrical signal to an electrical signal processor 1500.

The electro-optic modulator or optoelectronic integrated circuit may be applied to various electronic devices required light modulation, for example, optical communication devices. The electronic device may include various network equipment, mobile communications, computers, mobile phones, video phones, laptops, digital broadcasting terminals, e-books, navigation, medical devices, augmented reality (AR), virtual reality (VR), artificial intelligence (AI) devices, Internet of Things (IoT) devices, drones, security devices, or automotive electrical components, but is not limited thereto.

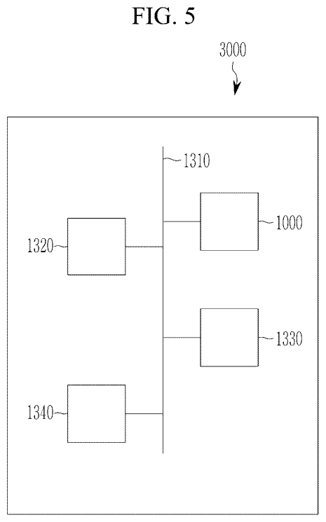

FIG. 5 is a schematic view illustrating an example of a configuration diagram of an electronic device according to some example embodiments.

Referring to FIG. 5, the electronic device 3000 may further include a bus 1310, a processor 1320, a memory 1330, and at least one additional device 1340, in addition to the aforementioned constituent elements. Information of the aforementioned optoelectronic integrated circuit 1000, processor 1320, memory 1330, and at least one additional device 1340 may be transmitted to each other through the bus 1310.

The processor 1320 may include one or more processing circuitry such as a hardware including logic circuits; a hardware/software combination such as processor-implemented software; or any combination thereof. For example, the processing circuitry may be a central processing unit (CPU), an arithmetic logic unit (ALU), a digital signal processor, a microcomputer, a field programmable gate array (FPGA), System-on-Chip (SoC), a programmable logic unit, a microprocessor, an application-specific integrated circuit (ASIC), and the like. As an example, the processing circuitry may include a non-transitory computer readable storage device. The processor 1320 may control, for example, the display operation of the optoelectronic integrated circuit 1000.

The memory 1330 may store an instruction program, and the processor 1320 may perform a function related to the optoelectronic integrated circuit 1000 by executing the stored instruction program.

The one or more additional devices 1340 may be one or more communication interfaces (e.g., wireless communication interfaces, wired interfaces), user interfaces (e.g., keyboard, mouse, buttons, etc.), power supply and/or power supply interfaces, or any combination thereof.

The units and/or modules described herein may be implemented using hardware constituent elements and software constituent elements. For example, the hardware constituent elements may include microphones, amplifiers, band pass filters, audio-to-digital converters, and processing devices. The processing device may be implemented using one or more hardware devices configured to perform and/or execute program code by performing arithmetic, logic, and input/output operations. The processing device may include a processor, a controller and an arithmetic logic unit, a digital signal processor, a microcomputer, a field programmable array, a programmable logic unit, a microprocessor, or any other device capable of responding to and executing instructions. The processing device may access, store, operate, process, and generate data in response to execution of an operating system (OS) and one or more software running on the operating system.

The software may include a computer program, a code, an instruction, or any combination thereof, and may transform a processing device for a special purpose by instructing and/or configuring the processing device independently or collectively to operate as desired. The software and data may be implemented permanently or temporarily as signal waves capable of providing or interpreting instructions or data to machines, parts, physical or virtual equipment, computer storage media or devices, or processing devices. The software may also be distributed over networked computer systems so that the software may be stored and executed in a distributed manner. The software and data may be stored by one or more non-transitory computer readable storage devices.

The method according to the foregoing embodiments may be recorded in a non-transitory computer readable storage device including program instructions for implementing various operations of the aforementioned embodiments. The storage device may also include program instructions, data files, data structures, and the like alone or in combination. The program instructions recorded in the storage device may be specially designed for the present embodiment or may be known to those skilled in computer software and available for use. Examples of non-transitory computer-readable storage devices may include magnetic media such as hard disks, floppy disks, and magnetic tapes; optical media such as CD-ROM discs, DVDs and/or blue-ray discs; magneto-optical media such as optical disks; and a hardware device configured to store and execute program instructions such as ROM, RAM, flash memory, and the like. The aforementioned device may be configured to operate as one or more software modules to perform the operations of the aforementioned embodiments.

Hereinafter, example embodiments are illustrated in more detail with reference to examples. However, these are non-limiting examples and the present scope is not limited thereto.

### Synthesis Examples: Synthesis of Electro-Optic Molecules

### Synthesis Example 1

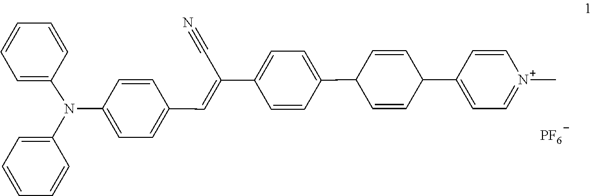

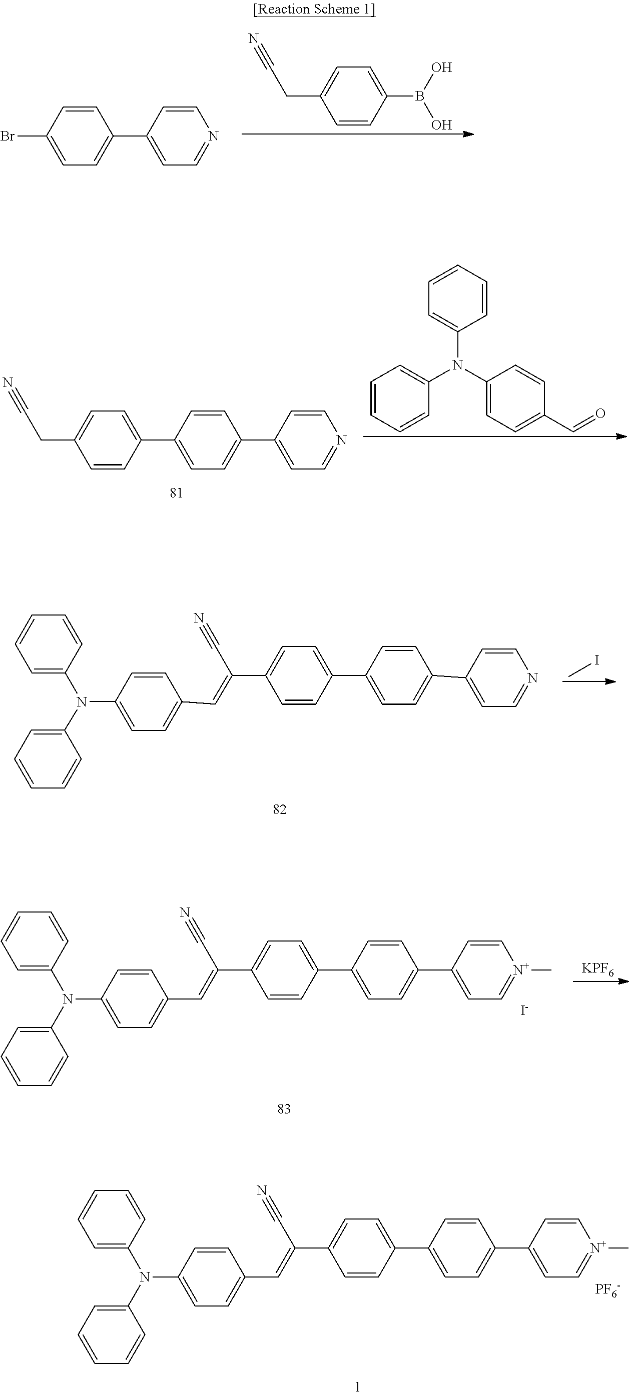

### (1) Synthesis of 2-(4′-(pyridin-4-yl)-[1,1′-biphenyl]-4-yl) acetonitrile (S1)

4-(4-bromophenyl)pyridine (1.04 g, 4.44 mmol) and (4-(cyanomethyl)phenyl) boronic acid (0.86 g, 5.32 mmol) are dissolved in 30 ml of a mixed solvent toluene/ethanol in a ratio of 1:1 v/v and then, stirred. Subsequently, 10 ml of a 2N K₂CO₃ aqueous solution is added thereto, and tetrakis(triphenylphosphine)palladium (0) (0.25 g, 0.2 mmol) is further added thereto and then, stirred at 80° C. for 24 hours. After cooling to room temperature, an organic solvent layer is separated therefrom by using distilled water and dichloromethane and then, treated with MgSO₄ to remove moisture remaining therein. Subsequently, S1 (0.72 g) in the form of solid powder is obtain by performing silica gel column chromatography and recrystallization. The yield is 60%.

### (2) Synthesis of 2-(4′-(pyridin-4-yl)-[1,1′-biphenyl]-4-yl) acetonitrile (S2)

S1 (0.72 g, 2.66 mmol) and 4-(diphenylamino)benzaldehyde (0.80 g, 2.93 mmol) are dissolved in 30 ml of an ethanol solution and then, stirred. Subsequently, potassium t-butoxide (0.60 g, 5.33 mmol) is added thereto and then, stirred at 55° C. for 2 hours. After cooling to room temperature, an organic solvent layer is separated therefrom by using distilled water and dichloromethane and then, treated with MgSO₄ to remove moisture remaining therein. Subsequently, silica gel column chromatography and recrystallization are performed to obtain S2 (0.44 g) in the form of solid powder. The yield is 31%.

### (3) Synthesis of (Z)-4-(4′-(1-cyano-2-(4-(diphenylamino)phenyl)vinyl)-[1,1′-biphenyl]-4-yl)-1-methylpyridin-1-ium iodide (S3)

S2 (0.44 g, 0.84 mmol) and iodomethane (0.078 ml, 1.26 mmol) are dissolved in a THF solvent (30 ml) and then, stirred at room temperature for 24 hours. Subsequently, recrystallization is performed to obtain S3 (0.20 g) in the form of solid powder. A yield thereof is 36%.

### (4) Synthesis of (Z)-4-(4′-(1-cyano-2-(4-(diphenylamino)phenyl)vinyl)-[1,1′-biphenyl]-4-yl)-1-methylpyridin-1-ium hexafluorophosphate (Compound 1)

S3 (0.20 g, 0.31 mmol) and an excessive amount of KPF₆ are dissolved in 30 ml of methanol and then, stirred at room temperature for 24 hours. Subsequently, recrystallization is performed to obtain Compound 1 (0.19 g) (electro-optic molecule) in the form of solid powder. The yield is 90%.

1H NMR (500.1 MHz, CDCl3/DMSO-d6) δ [ppm]: 8.72 (d, 2H), 8.02 (d, 2H), 7.70 (d, 2H), 7.60 (d, 2H), 7.55-7.46 (m, 6H), 7.29-7.26 (m, 2H), 7.05 (t, 4H), 6.89-6.86 (m, 5H), 6.76 (d, 2H), 4.20 (s, 3H).

HRMS (ESI+) calculated for C39H30N3+ [M]+: 540.24; measured: 539.92.

### Reference Synthesis Example 1

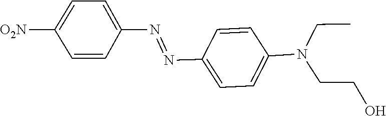 [SMILES: CCN(CCO)c1ccc(/N=N/c2ccc([N+](=O)[O-])cc2)cc1]

Disperse Red 1 reagent (electro-optic molecule) is purchased from Sigma-Aldrich Corporation.

### Reference Synthesis Example 2

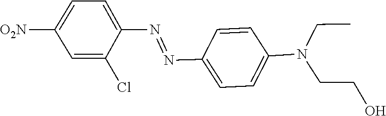 [SMILES: CCN(CCO)c1ccc(/N=N/c2ccc([N+](=O)[O-])cc2Cl)cc1]

Disperse Red 13 reagent (electro-optic molecule) is purchased from Sigma-Aldrich Corporation.

### Evaluation I

The electro-optic molecules according to Synthesis Example and Reference Synthesis Examples are evaluated with respect to electrical characteristics.

The electrical characteristics are evaluated from HOMO energy levels, LUMO energy levels, and energy bandgaps, which may be calculated by Turbomole using a B3LYP/DGDZVP basis set.

The results are shown in Table 1.

TABLE 1

| | HOMO (eV) | LUMO (eV) | Eg (eV) |
|---|---|---|---|
| Synthesis Example 1 | 6.85 | 5.88 | 0.97 |
| Reference Synthesis Example 1 | 5.46 | 3.53 | 1.93 |
| Reference Synthesis Example 2 | 5.91 | 3.19 | 2.72 |

Eg: energy bandgap

### Evaluation II

The electro-optic molecules according to Synthesis Example and Reference Synthesis Examples are evaluated with respect to electro-optic properties.

The electro-optic properties of the electro-optic molecules are evaluated from a dipole moment (μ), a polarization rate (α), and hyperpolarizability (β).

The dipole moment (μ) and the polarization rate (α) may be calculated by using B3LYP/DGDZVP, and the hyperpolarizability (β) may be calculated by using B3LYP/DGDZVP and M06-2X/DEF2TZVP.

The results are shown in Table 2.

TABLE 2

| | μ (Debye) | α (a.u.) | β (a.u.) |
|---|---|---|---|
| Synthesis Example 1 | 41.55 | 932.8 | 1952 |
| Reference Synthesis Example 1 | 9.11 | 325.1 | −142 |
| Reference Synthesis Example 2 | 11.8 | 346.6 | −151 |

Referring Table 2, it may be confirmed that the electro-optic molecules according to Synthesis Example 1 exhibits higher dipole moment, polarization rate, and hyperpolarizability than the electro-optic molecules according to Reference Synthesis Examples.

### EXAMPLES

### Example 1

ITO is deposited on a glass substrate to form a 100 nm-thick lower electrode (anode). On the lower electrode, aluminum oxide (Al₂O₃) is thermally deposited to form a 40 nm-thick lower auxiliary layer. Subsequently, electro-optic molecules according to Synthesis Example 1 and polymethylmethacrylate (PMMA) (T_{g}: 105 to 125° C.) are blended in a ratio of 10:90 wt % and then, spin-coated on the lower auxiliary layer at about 2000 rpm for 60 seconds and heated at about 80° C. for 24 hours to form a 1000 nm-thick electro-optic active layer. On the electro-optic active layer, silver (Ag) is deposited to form a 100 nm-thick upper electrode (cathode). Subsequently, an electric field of 60 V is applied between the lower and upper electrodes, and the electro-optic active layer is heat-treated at 85° C. on a hot plate for 5 minutes to align the electro-optic molecules in the electro-optic active layer in one direction along a thickness direction of the electro-optic active layer. After cooling the electro-optic active layer to room temperature, the electric field is removed to manufacture an electro-optic modulator.

### Example 2

An electro-optic modulator is manufactured in the same manner as in Example 1 except that a 500 nm-thick electro-optic active layer is formed instead of the 1000 nm-thick electro-optic active layer.

### Reference Example 1

An electro-optic modulator is manufactured in the same manner as in Example 1 except that the electro-optic molecules according to Reference Synthesis Example 1 is used instead of electro-optic molecules according to Synthesis Example 1.

### Reference Example 2

An electro-optic modulator is manufactured in the same manner as in Example 1 except that the electro-optic molecules according to Reference Synthesis Example 2 is used instead of electro-optic molecules according to Synthesis Example 1.

### Evaluation III

The electro-optic modulators according to Examples and Reference Examples are evaluated with respect to an electro-optic coefficient.

The linear electro-optic coefficient (r₃₃) is measured by using the simple reflection technique published by Teng et al., wherein a laser at a wavelength of 1550 nm is used as a light source, and each polarized thin membrane sample may be measured under the condition of a wavelength modulation voltage of 1 V at about 10 kHz.

$${r}_{33} = \frac{3 λ {I}_{m}}{4 {πV}_{m} {I}_{c} {n}^{2}} \frac{{( {n}^{2} - {sin}^{2} θ )}^{\frac{3}{2}}}{( {n}^{2} - 2 {sin}^{2} θ )} \frac{1}{{sin}^{2} θ} \qquad [ Relationship Equation 1 ]$$

In Relationship Equation 1,
Iₘ is a half of a voltage of amplified maximum modulation,
Vₘ is a voltage modulated from each sample, and
I_{c} is a median value of peak brightness of reflected light.

The results are shown in Table 3.

TABLE 3

| | r₃₃ (pm/V) |
|---|---|
| Example 1 | 16 |
| Example 2 | 62 |
| Reference Example 1 | 1 |
| Reference Example 2 | 3 |

Referring to Table 3, it may be confirmed that the electro-optic modulators according to Examples exhibit large electro-optic coefficients (r₃₃), compared with those according to Reference Examples. Accordingly, the electro-optic modulators including the compound according to Synthesis Example may exhibit large refractive index changes due to the electric field, compared with the electro-optic modulators including the compound according to Reference Synthesis Examples, and thus may be expected to exhibit improved electro-optic effects.

(1) A compound represented by Chemical Formula 1 is provided.

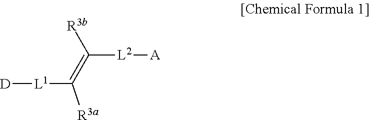

In Chemical Formula 1, A, D, L¹, L², R¹, R², R^{3a}, and R^{3b} are the same as described above.

(2) A in Chemical Formula 1 may be a group represented by any one of Chemical Formulas 1a to 1c.

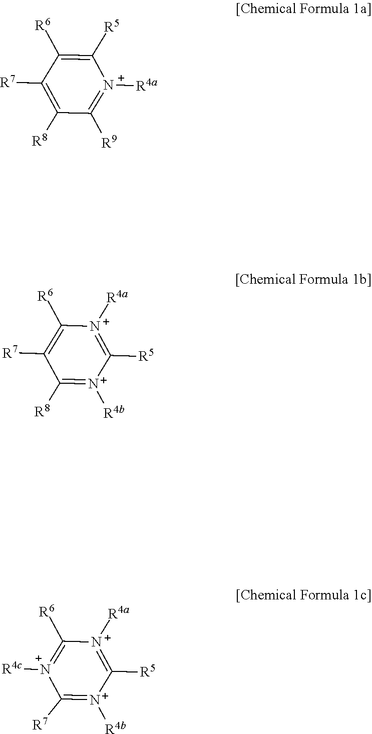

In Chemical Formulas 1a to 1c, R^{4a}, R^{4b}, R^{4c}, and R⁵ to R⁹ are the same as described above.

(3) L¹ and L² of Chemical Formula 1 may each independently be a single bond, a substituted or unsubstituted ethylene group, a substituted or unsubstituted propylene group, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene group, a substituted or unsubstituted dibenzotellurophenylene group, a fused ring thereof, or any combination thereof.

(4) R¹ and R² in Chemical Formula 1 may each independently be hydrogen, deuterium, a substituted or unsubstituted C1 to C20 alkyl group, a substituted or unsubstituted C1 to C20 alkylene group, a substituted or unsubstituted phenyl group, a substituted or unsubstituted phenylene group, a substituted or unsubstituted biphenyl group, a substituted or unsubstituted biphenylene group, a substituted or unsubstituted naphthyl group, a substituted or unsubstituted naphthylene group, or any combination thereof, wherein the “substituted” refers to replacement of at least one hydrogen by a hydroxy group, a silyl group, an amino group, a C1 to C30 alkyl group, a C6 to C30 aryl group, a C1 to C30 alkoxy group, a substituted or unsubstituted C1 to C30 alkylthio group, a C1 to C30 alkylseleno group, or any combination thereof, and R¹ and R² are each independently present, or two or three adjacent may be linked to each other to form a ring.

(5) The compound may be represented by any one of Chemical Formulas 1-1 to 1-6.

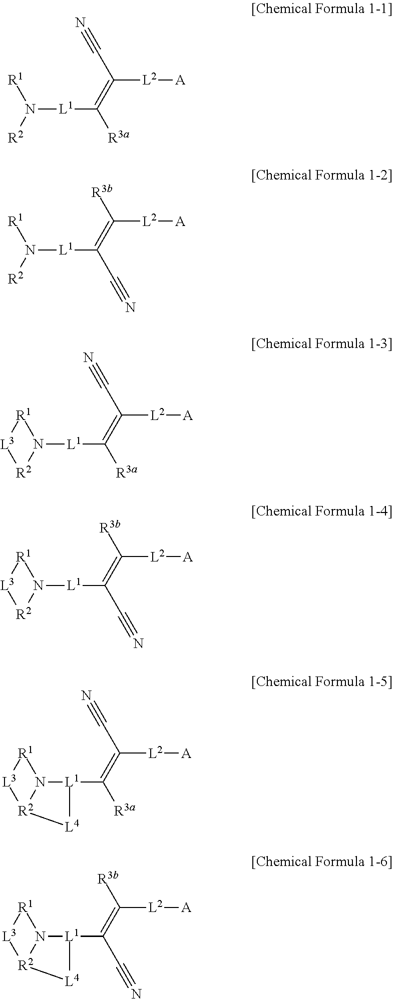

In Chemical Formulas 1-1 to 1-6, A, L¹ to L⁴, R^{3a}, R^{3b}, R¹, and R² are the same as described above.

(6) A in Chemical Formula 1 may be a group represented by any one of Chemical Formulas 1a to 1c.

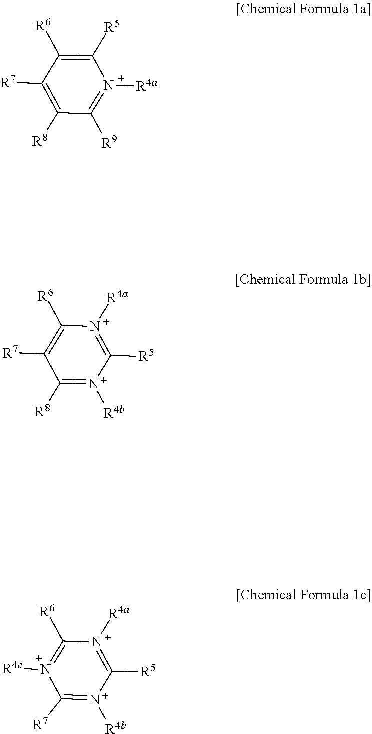

In Chemical Formulas 1a to 1c, R^{4a}, R^{4b}, R^{4c}, and R⁵ to R⁹ are the same as described above.

(7) The compound may further include a counterpart anion.

(8) An electro-optical material including the compound represented by Chemical Formula 1 and a polymer is provided.

(9) An absorbance in the wavelength range of about 1300 nm to about

1700 nm of the polymer may be less than about 10%.

(10) The compound may further include a counterpart anion.

(11) The compound and the polymer may be included in a weight ratio of about 1:99 to about 99:1.

(12) An electro-optic modulator includes: a first electrode, a second electrode, and an electro-optic active layer between the first electrode and the second electrode, wherein the electro-optic active layer includes the compound represented by Chemical Formula 1.

(13) The compound may further include a counterpart anion, and the electro-optic active layer may further include a polymer.

(14) An absorbance in the wavelength range of about 1300 nm to about 1700 nm of the polymer may be less than about 10%, and the electro-optic active layer may include the compound and the polymer in a weight ratio of about 1:99 to about 99:1.

(15) The compound may be aligned in one direction.

(16) The electro-optic modulator may further include a substrate including silicon, germanium, an III-V compound semiconductor, an II-VI compound semiconductor, or any combination thereof.

(17) A method of manufacturing an electro-optic modulator includes: forming a first electrode, forming an electro-optic active layer on the first electrode, the electro-optic active layer including the compound represented by Chemical Formula 1, forming a second electrode on the electro-optic active layer, and performing a poling process to align the compound.

(18) The performing of the poling process may include heat treating at a temperature of about +50° C. of the glass transition temperature of the polymer, aligning the compound by applying a voltage, and cooling.

(19) The compound may be aligned along a thickness direction of the electro-optic active layer.

(20) An optoelectronic integrated circuit includes: a substrate, an electrical signal processor on the substrate and configured to perform electrical signal processing, a light source on the substrate, an electro-optic modulator on the substrate and configured to output an electrical signal into a modulated optical signal using an electrical signal output from the electrical signal processor and light supplied from the light source, and a photodetector on the substrate and configured to detect the modulated optical signal and to convert the modulated optical signal into an electrical signal, wherein the electro-optic modulator includes a first electrode and a second electrode, and an electro-optic active layer between the first electrode and the second electrode, and the electro-optic active layer includes the compound represented by Chemical Formula 1.

(21) The substrate may include a silicon substrate, a germanium substrate, a silicon germanium substrate, an III-V compound semiconductor substrate, an II-VI compound semiconductor substrate, an SOI substrate, a SOG substrate, or any combination thereof, and the light source may supply light having a wavelength of about 1000 nm to about 1700 nm.

(22) An optical communication device includes: an optoelectronic integrated circuit including an electrical signal processor, an electro-optic modulator, a photodetector, and a light source integrated therein, wherein the electro-optic modulator includes the compound represented by Chemical Formula 1.

One or more of the elements disclosed above may include or be implemented in processing circuitry such as hardware including logic circuits; a hardware/software combination such as a processor executing software; or a combination thereof. For example, the processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

While this disclosure has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

What is claimed is:

1. A compound represented by Chemical Formula 1: 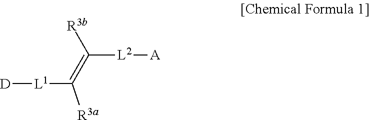
wherein, in Chemical Formula 1,
A is a substituted or unsubstituted nitrogen cation-containing ring group,
D is —NR¹R²,
L¹ and L² are each independently a single bond, a substituted or unsubstituted C1 to C30 alkylene group, a substituted or unsubstituted C2 to C30 alkenylene group,
a substituted or unsubstituted C6 to C30 arylene group, a substituted or unsubstituted divalent C2 to C30 heterocyclic group, a fused ring thereof, or any combination thereof,
R¹, R², R^{3a}, and R^{3b} are each independently hydrogen, deuterium, a substituted or unsubstituted C1 to C20 alkyl group, a substituted or unsubstituted C1 to C20 alkylene group, a substituted or unsubstituted C2 to C30 alkenyl group, a substituted or unsubstituted C2 to C30 alkenylene group, a substituted or unsubstituted C1 to C30 alkoxy group, a substituted or unsubstituted C1 to C30 alkylthio group, a substituted or unsubstituted C6 to C30 aryl group, a substituted or unsubstituted C6 to C30 arylene group, a substituted or unsubstituted C3 to C30 heterocyclic group, a halogen, a hydroxyl group, a cyano group, or any combination thereof,
at least one of R^{3a} or R^{3b} is a cyano group, and
R¹, R², R^{3a}, R^{3b}, and L¹ are each independently present or two or three adjacent R¹, R², R^{3a}, R^{3b}, and L¹ are linked to each other to form a ring.

2. The compound of claim 1, wherein A in Chemical Formula 1 is a group represented by any one of Chemical Formulas 1a to 1c: 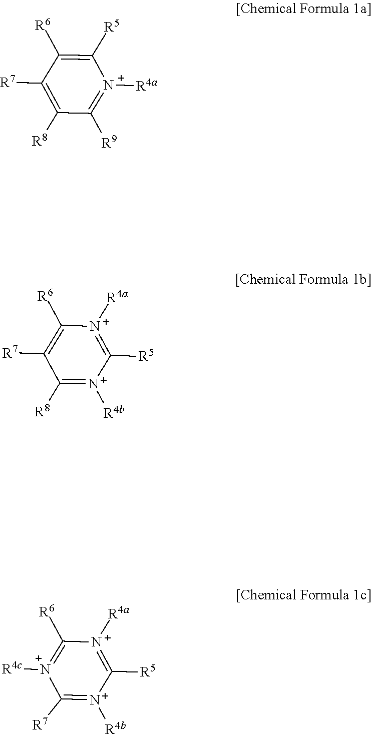
wherein, in Chemical Formulas 1a to 1c,
R^{4a}, R^{4b}, R^{4c}, and R⁵ to R⁹ are each independently hydrogen, deuterium, a substituted or unsubstituted C1 to C20 alkyl group, a substituted or unsubstituted C2 to C30 alkenyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heterocyclic group, a halogen, a hydroxyl group, a cyano group, or any combination thereof, or a linking point with Chemical Formula 1,
R^{4a}, R^{4b}, R^{4c}, and R⁵ to R⁹ are each independently present, or two or three adjacent R^{4a}, R^{4b}, R^{4c}, and R⁵ to R⁹ are linked to each other to form a ring, and
any one of R^{4a} and R⁵ to R⁹ of Chemical Formula 1a, any one of R^{4a}, R^{4b}, and R⁵ to R⁸ of Chemical Formula 1b, and any one of R^{4a}, R^{4b}, R^{4c} and R⁵ to R⁷ of Chemical Formula 1c are each a linking point with Chemical Formula 1.

3. The compound of claim 1, wherein L¹ and L² are each independently a single bond, a substituted or unsubstituted ethylene group, a substituted or unsubstituted propylene group, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene group, a substituted or unsubstituted dibenzotellurophenylene group, a fused ring thereof, or any combination thereof.

4. The compound of claim 1, wherein
R¹ and R² are each independently hydrogen, deuterium, a substituted or unsubstituted C1 to C20 alkyl group, a substituted or unsubstituted C1 to C20 alkylene group, a substituted or unsubstituted phenyl group, a substituted or unsubstituted phenylene group, a substituted or unsubstituted biphenyl group, a substituted or unsubstituted biphenylene group, a substituted or unsubstituted naphthyl group, a substituted or unsubstituted naphthylene group, or any combination thereof,
the “substituted” refers to replacement of at least one hydrogen by a hydroxy group, a silyl group, an amino group, a C1 to C30 alkyl group, a C6 to C30 aryl group, a C1 to C30 alkoxy group, a substituted or unsubstituted C1 to C30 alkylthio group, a C1 to C30 alkylseleno group, or any combination thereof, and
R¹ and R² are each independently present or two or three adjacent ones are linked to each other to form a ring.

5. The compound of claim 1, wherein the compound is represented by any one of Chemical Formulas 1-1 to 1-6: 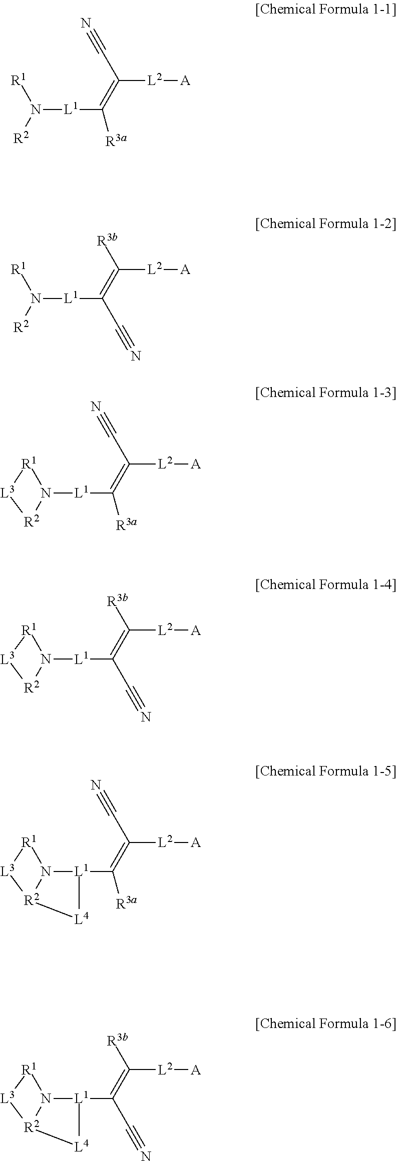
wherein, in Chemical Formulas 1-1 to 1-6,
A is a substituted or unsubstituted nitrogen cation-containing ring group,
L¹ and L² are each independently a single bond, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted furanylene group, a substituted or unsubstituted thiophenylene group, a substituted or unsubstituted selenophenylene group, a substituted or unsubstituted tellurophenylene group, a substituted or unsubstituted benzofuranylene group, a substituted or unsubstituted benzothiophenylene group, a substituted or unsubstituted benzoselenophenylene group, a substituted or unsubstituted benzotellurophenylene group, a substituted or unsubstituted dibenzofuranylene group, a substituted or unsubstituted dibenzothiophenylene group, a substituted or unsubstituted dibenzoselenophenylene group, a substituted or unsubstituted dibenzotellurophenylene group, a fused ring thereof, or any combination thereof,
R^{3a} or R^{3b} is hydrogen, deuterium or a substituted or unsubstituted C1 to C20 alkyl group,
R¹ and R² are each independently hydrogen, deuterium, a substituted or unsubstituted C1 to C20 alkyl group, a substituted or unsubstituted C1 to C20 alkylene group, a substituted or unsubstituted phenyl group, a substituted or unsubstituted phenylene group, a substituted or unsubstituted biphenyl group, a substituted or unsubstituted biphenylene group, a substituted or unsubstituted naphthyl group, a substituted or unsubstituted naphthylene group, or any combination thereof,
L³ and L⁴ are each independently a single bond, —O—, —S—, —Se—, —Te—, —NR^{a1}—, —BR^{a2}—, —SiR^{b}R^{c}—, —GeR^{d}R^{e}—, —(CR^{f}R^{g})ₙ₁—, —(C(R^{m})═C(Rⁿ))—, or —(C(R^{p})═N))—,
wherein R^{a1}, R^{a2}, R^{b}, R^{c}, R^{d}, R^{e}, R^{f}, R^{g}, R^{m}, Rⁿ, and R^{p} are each independently hydrogen, deuterium, a substituted or unsubstituted C1 to C20 alkyl group, a substituted or unsubstituted C1 to C20 alkoxy group, a substituted or unsubstituted C6 to C20 aryl group, or a halogen,
R^{a1}, R^{a2}, R^{b}, R^{c}, R^{d}, R^{e}, R^{f}, R^{g}, R^{m}, Rⁿ, and R^{p} are each independently present or two adjacent ones are linked to each other to form a ring, and
n1 is 1 or 2.

6. The compound of claim 5, wherein A in Chemical Formulas 1-1 to 1-6 is a group represented by any one of Chemical Formulas 1a to 1c: 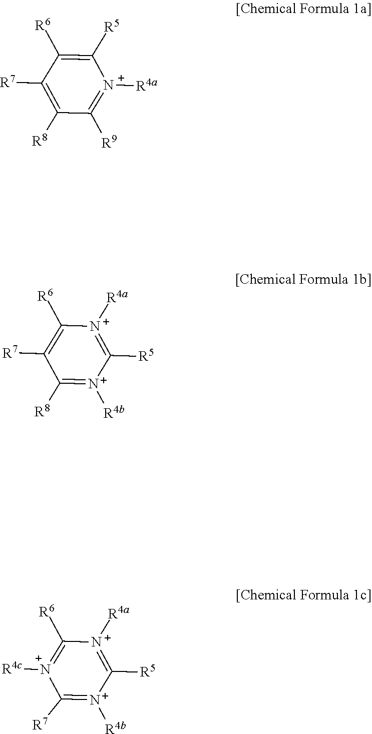
wherein, in Chemical Formulas 1a to 1c,
R^{4a}, R^{4b}, R^{4c}, and R⁵ to R⁹ are each independently hydrogen, deuterium, a substituted or unsubstituted C1 to C20 alkyl group, a substituted or unsubstituted C2 to C30 alkenyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heterocyclic group, a halogen, a hydroxyl group, a cyano group, or any combination thereof, or a linking point with Chemical Formula 1,
R^{4a}, R^{4b}, R^{4c}, and R⁵ to R⁹ are each independently present or two or three adjacent R^{4a}, R^{4b}, R^{4c}, and R⁵ to R⁹ are linked to each other to form a ring, and
any one of R^{4a} and R⁵ to R⁹ of Chemical Formula 1a, any one of R^{4a}, R^{4b}, and R⁵ to R⁸ of Chemical Formula 1b, and any one of R^{4a}, R^{4b}, R^{4c} and R⁵ to R⁷ of Chemical Formula 1c are each a linking point with Chemical Formula 1.

7. The compound of claim 1, further comprising:
a counterpart anion.

8. An electro-optic material, comprising
electro-optic molecules, each of the electro-optic molecules including the compound of claim 1, and
a polymer.

9. The electro-optic material of claim 8, wherein an absorbance in a wavelength range of 1300 nm to 1700 nm of the polymer is less than 10%.

10. The electro-optic material of claim 8, wherein the electro-optic molecules further comprise a counterpart anion.

11. The electro-optic material of claim 8, wherein the electro-optic molecules and the polymer are included in a weight ratio of 1:99 to 99:1.

12. An electro-optic modulator, comprising
a first electrode;
a second electrode; and
an electro-optic active layer between the first electrode and the second electrode,
wherein the electro-optic active layer comprises electro-optic molecules, and
each of the electro-optic molecules include a compound represented by Chemical Formula 1: 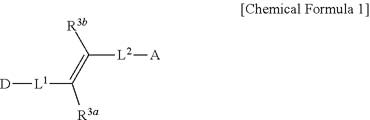
wherein, in Chemical Formula 1,
A is a substituted or unsubstituted nitrogen cation-containing ring group,
D is —NR¹R²,
L¹ and L² are each independently a single bond, a substituted or unsubstituted C1 to C30 alkylene group, a substituted or unsubstituted C2 to C30 alkenylene group, a substituted or unsubstituted C6 to C30 arylene group, a substituted or unsubstituted divalent C2 to C30 heterocyclic group, a fused ring thereof, or any combination thereof,
R¹, R², R^{3a}, and R^{3b} are each independently hydrogen, deuterium, a substituted or unsubstituted C1 to C20 alkyl group, a substituted or unsubstituted C1 to C20 alkylene group, a substituted or unsubstituted C2 to C30 alkenyl group, a substituted or unsubstituted C2 to C30 alkenylene group, a substituted or unsubstituted C1 to C30 alkoxy group, a substituted or unsubstituted C1 to C30 alkylthio group, a substituted or unsubstituted C6 to C30 aryl group, a substituted or unsubstituted C6 to C30 arylene group, a substituted or unsubstituted C3 to C30 heterocyclic group, a halogen, a hydroxyl group, a cyano group, or any combination thereof,
at least one of R^{3a} or R^{3b} is a cyano group, and
R¹, R², R^{3a}, R^{3b}, and L¹ are each independently present or two or three adjacent R¹, R², R^{3a}, R^{3b}, and L¹ are linked to each other to form a ring.

13. The electro-optic modulator of claim 12, wherein the compound further comprises a counterpart anion.

14. The electro-optic modulator of claim 12, wherein the electro-optic active layer further comprises a polymer.

15. The electro-optic modulator of claim 14, wherein an absorbance in a wavelength range of 1300 nm to 1700 nm of the polymer is less than 10%.

16. The electro-optic modulator of claim 14, wherein the electro-optic active layer comprises the electro-optic molecules and the polymer in a weight ratio of 1:99 to 99:1.

17. The electro-optic modulator of claim 12, wherein the electro-optic molecules are aligned in one direction.

18. The electro-optic modulator of claim 12, further comprising:
a substrate, wherein
the substrate includes silicon, germanium, an III-V compound semiconductor substrate, an II-VI compound semiconductor, or any combination thereof.

19. An optoelectronic integrated circuit, comprising
a substrate,
an electrical signal processor on the substrate, the electrical signal processor being configured to perform electrical signal processing to output an electrical signal,
a light source on the substrate; and
an electro-optic modulator on the substrate, the electro-optic modulator being configured to output a modulated optical signal using the electrical signal output from the electrical signal processor and light supplied from the light source;
a photodetector on the substrate, the photodetector being configured to detect the modulated optical signal and convert the modulated optical signal into a converted electrical signal,
wherein the electro-optic modulator comprises a first electrode, a second electrode, and an electro-optic active layer between the first electrode and the second electrode,
wherein the electro-optic active layer comprises electro-optic molecules and each of the electro-optic molecules is a compound represented by Chemical Formula 1, 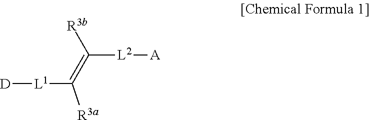
wherein, in Chemical Formula 1,
A is a substituted or unsubstituted nitrogen cation-containing ring group,
D is —NR¹R²,
L¹ and L² are each independently a single bond, a substituted or unsubstituted C1 to C30 alkylene group, a substituted or unsubstituted C2 to C30 alkenylene group, a substituted or unsubstituted C6 to C30 arylene group, a substituted or unsubstituted divalent C2 to C30 heterocyclic group, a fused ring thereof, or any combination thereof,
R¹, R², R^{3a}, and R^{3b} are each independently hydrogen, deuterium, a substituted or unsubstituted C1 to C20 alkyl group, a substituted or unsubstituted C1 to C20 alkylene group, a substituted or unsubstituted C2 to C30 alkenyl group, a substituted or unsubstituted C2 to C30 alkenylene group, a substituted or unsubstituted C1 to C30 alkoxy group, a substituted or unsubstituted C1 to C30 alkylthio group, a substituted or unsubstituted C6 to C30 aryl group, a substituted or unsubstituted C6 to C30 arylene group, a substituted or unsubstituted C3 to C30 heterocyclic group, a halogen, a hydroxyl group, a cyano group, or any combination thereof,
at least one of R^{3a} or R^{3b} is a cyano group, and
R¹, R², R^{3a}, R^{3b}, and L¹ are each independently present or two or three adjacent R¹, R², R^{3a}, R^{3b}, and L¹ are linked to each other to form a ring.

20. The optoelectronic integrated circuit of claim 19, wherein
the substrate comprises a silicon substrate, a germanium substrate, a silicon germanium substrate, an III-V compound semiconductor substrate, an II-VI compound semiconductor substrate, an SOI substrate, a SOG substrate, or any combination thereof, and
the light source is configured to supply light of a wavelength within 1000 nm to 1700 nm.
